(19) United States
(12) Patent Application Publication
(10) Pub. No.: US 2007/0111973 A1
(43) Pub. Date: May 17, 2007

(54) TREATMENT OF TUMOURS

(76) Inventors: Tomas Hagstrom, Svartmala Gard (SE); Peter Soderkvist, Lindnegatan 39 (SE); Jan-Erling Backvall, Egils-vagen 2 (SE)

Correspondence Address: GAUTHIER & CONNORS, LLP, 225 FRANKLIN STREET, SUITE 2300, BOSTON, MA, 02110, US

(21) Appl. No.: 11/609,643

(22) Filed: Dec. 12, 2006

Related U.S. Application Data
(62) Division of application No. 10/658,125, filed on Sep. 9, 2003.
Application No. 10/658,125 is a continuation of application No. PCT/SE02/00443, filed on Mar. 11, 2002.

(30) Foreign Application Priority Data
Mar. 13, 2001 (SE) 0100857-2

(51) Int. Cl. A61K 31/56

(52) U.S. Cl. 514/169; 514/178

(57) ABSTRACT

The present invention refers to steroid derivatives for use as medicaments. More specifically, the invention also relates to the use of a steroid derivative of 5-androstene-, 5-pregnenolone or corresponding saturated derivatives (androstane- or pregnane-) in the manufacture of a medicament for the treatment of a benign and/or malignant tumour, which medicament is capable of interrupting disturbances in Wut-signaling, such as cell-cycle arrest in G1-phase, and/or providing an angiostatic effect. Examples of such steroid derivatives are -5-androstene-17-ol, androstane-17-ol-pregnane-17-ol or pregnane-17-ol derivatives. In a further aspect, the invention relates to a method of producing a medicament for the treatment of a benign and/or malignant tumour and/or an inflammatory condition comprising the steps of contacting 5-androstane-3B,17-dio 1 or androstane-3B-diol, an enzyme and a sulfotransferase to provide 5-androstene-17-ol-3B-sulfate or corresponding andros tane derivative (17-AEDS or 17-AADS); and mixing the 17-AEDS or 17-AADS so produced with a suitable carrier; whereby a medicament which is capable of acting as a ligand to peroxisome proliferators-activated receptor—(PPAR) is produced.

### TECHNICAL FIELD

The present invention relates to novel steroid derivatives, which are useful as medicaments. The invention also relates to the use of steroid derivatives in the manufacture of a medicament e.g. for the treatment of a benign and/or malignant tumour, such pharmaceutical compositions, as well as method for treating benign and malignant tumours.

### BACKGROUND

U.S. Pat. No. 5,912,240 (Loria) describes the steroid androstene-3β,17α-diol (17α-AED) and antitumoural effects through inhibiting growth and inducing apoptosis in all neoplastic cell lines. Apoptosis is demonstrated therein in vitro in three neoplastic myeloid cell lineages. In two breast cancer cell lines growth-inhibition is demonstrated. However, no apoptosis was demonstrated in the two breast cancer cell lines.

Nuclear receptor PPARγ is a transcription factor belonging to the steroid hormone receptor superfamily. Nuclear receptors link extracellular hormone signals to a transcriptional response. This is done through binding of the receptor to response elements located within promoter regions of target genes. Some nuclear receptors of this family exert only ligand-dependent effects, while others function in the absence of ligands. Members of the steroid hormone receptor superfamily includes glucocorticoid receptor and receptors for estrogens, androgens, progestins, thyroid hormone, retinoic acid, 9-cis-retinoic acid, peroxisome proliferators, vitamin D and ecdysone.

The receptor consists of six domains. Counting from the N-terminal A-F, where ligand-independent function (AF-1) resides in A/B and ligand-dependent function AF-2) in E. The C-domain is the DNA-binding domain (DBD).

Waxman (Role of metabolism in the activation of dehydro-epiandrosterone as a pertxisome proliferator, D J Waxman, J of Endocrinology, vol 150, suppl. September 1996) indirectly demonstrated ligand activity of 3β-sulfate of 5-androstene-3β,17β-diol to PPARα.

### SUMMARY OF THE INVENTION

One object of the invention is to provide a novel compound, which is capable of controlling cell cycle arrest and/or an angiostatic effect, which compound is useful as a medicament. This and other objects of the invention are achieved as described by the appended claims.

More specifically, the invention-relates to administrating a therapeutic dose of such a steroid to cause a down-regulation of cyclin D1 or β-catenin in instances where these factors are overexpressed and present a hindrance to therapy, to remove the cell-cycle block or to down-regulate pathologic vascularization. Examples of such instances are colorectal carcinomas which are mutated in the APC-gene in a majority of cases, which causes an upregulation of β-catenin, a minority of prostate cancers with increased expression of cyclin D1 or β-catenin as well as some mammary cancers with increased expression of β-catenin of phenotypic reasons. In the field of lymphomas there is an especially interesting subgroup, the mantle-cell lymphomas, known to be resistant to virtually all known treatment, which are characterized by their expression of cyclin D1.

The effects obtained according to the present invention can be used by them or combined with traditional cytostatic therapy or irradiation. In some instances it can be useful or necessary to combine the novel steroid according to the invention with ligands to nuclear receptors such as androgens, anti-androgens, estrogens, anti-estrogens, retinoic acid derivatives, deltanoids, levaxin etc. either to block an unwanted affinity of the steroid in question to an irrelevant nuclear receptor (such as affinity to progesterone, androgen or estrogen-receptors), to increase the efficacy of the treatment through repressing a competing process by providing a better substrate for the competing process than the steroid used (androgen receptor competing successfully with PPARγ for cofactor ARA70 and hence squelching unwanted PPARγ-activity).

The invention also deals with a novel PPARγ-ligand and PPARγ-ligand activity of 3β-steroid sulfates, how to promote or to block such activity, and advantageous applications thereof. The invention also encompasses the new steroids as such and their effects.

### BRIEF DESCRIPTION OF THE DRAWINGS

FIG. 1 is a picture of undifferentiated control and tumour treated with 17α+17β-AED.

FIG. 2 shows Western blots as discussed in relation to Table IV.

FIG. 3 illustrates effects on VEGF of 17α-AED, 17β-AED, 17αOH-pregnenolone and 5-androstene-3β,7β,17α-triol.

FIG. 4 shows Western blots, illustrating effects of 17α-AED, 17αOH-pregnenolone -and 17β-AED on expression of β-catenin, cyclin D1 and COX-2 in Dunning AT-1, rat prostate tumours.

FIG. 5 shows a comparison of proportion of cells in G1, S-phase in untreated controls (CA-CD) and after treatment with 17α-AED (alfaA-D) illustrated by representative examples.

FIG. 6 illustrates how 17α-AED,17OH-pregnenolone, 5-androstene-3β,7β,17α and their respective 3β-sulfates were investigated for PPARγ-ligand activity in a ligand-induced coactivator assay.

### DETAILED DESCRIPTION OF THE INVENTION

In a first aspect, the present invention relates to a novel steroid derivative for use as a medicament. The derivative according to the invention is described by either one of the general formulas (I) and (II) below, the only difference between said formulas being the nature of the bond between carbon atoms nos. 5 and 6, which is a double bond in formula (I), as shown below:

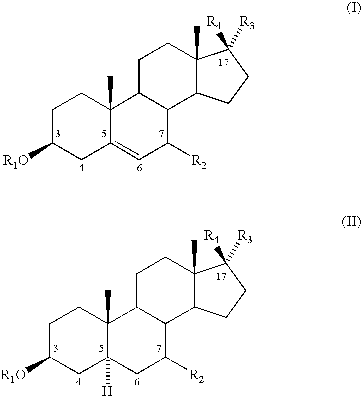

wherein the steroids have R₁O-substituents in 3β-position and R₃-substituents in 17α-position and optional substituents in 7 and 17β-position.

R₁ on oxygen at position 3 and can be: (I) a hydrogen atom, (ii) an NO₂, an SO3H, an —OP(OH)3, an acyl group, or any other group forming an ester with an inorganic or organic acid, (iii) a protecting group, such as CH₃, CH₂OMe, CH₂O-alkyl (iv) any other aliphatic chain which can be straight or branched, saturated or unsaturated, substituted or unsubstituted, cyclic, including mixed cyclic and aliphatic substituents, saturated or aromatic or heterocyclic substituents containing up to 20 carbon atoms. Substituents may be selected from OH, halogen (F, Cl, Br, I), amino, alkylamino or dialkylamino.

Further, in a specific embodiment, R₁ can form ethers or esters with the steroid. R₂ can be hydrogen in formula (II) and can be hydrogen in formula (I) provided R₄is not H, or can be R′O in a or 3-position of the carbon number 7, where R′ independently of R₁ may be any substituent acceptable for R₁.

R₂ can also be ═O or ═S.

R₃ is always in 17α-position and may be an hydroxyl-group, an acyl-group or may be R″O, where R″ may be any other group forming an ether or an ester as described for R₁ or any other substituent acceptable for, but independent of R₁.

R₄ is always in 17β-position and can be a hydrogen atom, an alkyl group, an acyl-group, an alkoxy group, the latter of the formula R′″O, wherein R′″ may be any other group forming an ether or an ester or any other substituent acceptable for, but independent of R₁.

Further R₄ can be an acyl group, in which hydrogen or an alkoxy or alkyl group may be attached to the keto group.

In a specific embodiment R₄ can be acetyl (CH₃CO), as in 17OH-pregnenolone, where a methyl is attached to the keto-group. This keto-carbon numbered 20 could have any alkyl, alkenyl, aryl, including branched side chains or mixed aromatic and aliphatic side chains, including cyclic saturated hydrocarbons as well as heterocyclic rings or heteroaliphatic chains containing e.g. N, P, O, Si, F, S, Se, CN, halogens and containing up to 20 carbons.

In one embodiment, said steroid is, 17-hydroxy-pregnenolone (17α-OH), Δ-5-androstene-3β,17α-diol, Δ-5-androstene-7-oxo-3β, 17α-diol and/or 5-androstene-3β,7β,17α-triol.

In another embodiment said steroid is the corresponding pregnane- and/or androstane-derivatives.

In a specific embodiment, the above mentioned effects are independent of any direct apoptotic effect on the cells of said tumour.

The novel steroids according to the invention are useful in medicaments, which when administered to a patient in need of therapy is capable of providing one or more antitumoural effects through interference with the Wnt signaling pathway. Such medicaments are especially advantageous for the treatment of tumours where an overexpression of factors from this pathway due to a mutation in factors regulating this pathway or where a phenotypic overexpression occurs, where tumours have been shown to be resistent to some forms of conventional treatment.

In the most advantageous embodiment, a medicament which comprises one or more steroids according to the invention is for the treatment and/or prevention of a medical condition selected from the group consisting of colon malignancies with a genetic overexpression, cancers caused by a phenotypic upregulation of cyclin D1 and/or β-catenin (estrogen receptor-negative breast cancers), lung cancers, melanomas, mantle cell lymphomas and other B-cell lymphomas characterized by over-expression of cyclin D1, parathyroid adenomas and cancers, head and neck tumours of squamous cell origin, oesophagal tumours and tumours and other pathologic conditions dominated by a destructive neovascularisation (diabetic retinopathy, exsudative forms of macular degeneration, corneal neovascularisation, vascular-tumours as hemanglomas, malignant vascular tumours, midline granulomas and uncontrolled growth of scars as in keloid formation.

In a second aspect, the present invention relates to the use of a steroid derivative of 5-androstene-, 5-pregnenolone or corresponding saturated derivatives (androstane- or pregnane-) in the manufacture of a medicament for the treatment and/or prevention of a benign and/or malignant tumour, which medicament is capable of interrupting disturbances in Wnt-signaling, such as cell-cycle arrest in G1-phase, and/or providing an angiostatic effect.

In a specific embodiment, said steroid derivate is described by formula (I) or (II) as shown and defined above.

As mentioned above, in one embodiment, R₁, R′ and/or R″ of said formulas form one or more ether(s) and/or ester(s) with the steroid. Further, R₄ can be an acyl group, in which a hydrogen, or an alkoxy or alkyl group, is attached to the keto group. In a specific embodiment, R₄ is acetyl (CH₃CO), where a methyl is attached to the keto group, and this keto carbon in position 20 has an alkyl, alkenyl, aryl, including branched, side chain or a mixed aromatic and aliphatic side chain, including cyclic saturated hydrocarbons as well as heterocyclic rings or heteroaliphatic chains, such as those comprising N, P, O, Si, S, Se, CN, or one or more halogen and comprises up to 20 carbons.

2. In an advantageous embodiment, the steroid is selected from the group consisting of 17-hydroxy-pregnenolone (17α-OH), Δ-5-androstene-3β,17α-diol, Δ-5-androstene-3β,17α-diol-7-oxo, 5-androstene-3β,7β,17α-triol, 5-androstene-3β,7β,17α-triol and 5-androstene-3β,17α-diol-7-one, 5-androstane-3β,7β,17α-triol, 5-androstene-3β,7β,17α-triol, 5-androstane-3β,7α,17α-triol, and 5-androstane-3β,17α-diol.

One or more pregnane- and/or androstane-derivative corresponding to the steroid can be used In the manufacture of the medicament according to the invention.

The above discussed interruption is provided by downregulating an overexpression of cyclin D1 and β-catenin, and effects are advantageously essentially independent of any direct apoptotic effect on the cells of said tumour.

The medicament produced according to this aspect of the invention useful is for the treatment and/or prevention of one or more medical conditions selected from the group that consists of colon malignancies and other malignancies with a genotypic or phenotypic overexpression of factors belonging to the Wnt-signaling pathway, such as lung cancers, melanomas, breast cancers, mantle cell lymphomas and other lymphomas as well as a fraction of prostate cancers, characterized by an up-regulation of said factors, head and neck cancers of squamous cell origin, oesophagal cancers, parathyroid cancers or adenomas or other tumours characterized by a disturbance in Wnt-signaling; and conditions dominated by pathologic neovascularisation, such as diabetic retinopathy, exsudative forms of macular degeneration, corneal neovascularisation, and vascular tumours.

Concerning the potential usefulness of mentioned steroids in the context of prostate cancer a deregulation of Wnt-signaling exists also in a small subfraction of prostate cancer making steroids according to the invention useful, with the exceptions of

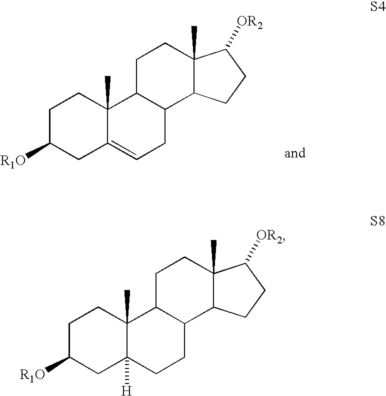

for the following reasons: Contrary to the Dunning AT-1, rat prostate cancer model which completely lacks androgen and estrogen-receptors (AR and ER), most human prostate cancers show an abundant expression of AR and usually also ERβ. In the androgen refractory cancers AR is even up-regulated. The influence of an up-regulated β-catenin on androgen-receptor (AR) transcription leads to increased AR transcriptional activity from present androgens as well as decreased ligand-specificity, which limits the usefulness of 17α-AED in this disease, since it is readily metabolized into epitestosterone—or into B) 17α-AED-3β-sulfate, both potential androgen-receptor-ligands, which are in turn easily converted into other known AR-ligands (see references in discussion).

In androgen-refractory progression AR is activated also by ligand-independent factors such as epidermal growth factor (EGF) and IL-6. EGF-receptor-activation in turn up-regulates expression of cyclin D1 (see references in discussion). The above described two compounds S4 and S8 are thus of potential value only in a small subfraction of human prostate cancers, expressing aberrant Wnt-signaling and their usefulness is further limited by their androgenic activity and their metabolisatlon to other androgens.

In cancers with a cell-cycle regulation defect due to aberrant Wnt-signaling as exemplified or in instances of destructive neovascularisation, such a process may be limited or stopped through treatment with a steroid according to the invention alone or as pre-surgical or pre-radiological treatment or in combination with cytotoxic drugs, interferons, cytokines or steroid hormones.

In a third aspect, the present invention relates to a method of producing a medicament for the treatment of a benign and/or malignant tumour, comprising the steps of

(a) contacting 5-androstene-3β,17α-diol or androstane-3β,17α-diol, a sulfate donor, 3′ phospho-adenosine-5′phosphosulphate (PAPS) and a sulphotransferase to provide 5-androstene-17α-ol-3β-sulfate (17α-AEDS) or androstane-17α-ol-3β-sulfate (17α-AADS); and

(b) combining the sulfated 17α-AEDS or 17α-AADS so produced with a suitable carrier; whereby a medicament which is capable of acting as a ligand to peroxisome proliferator-activated receptors (PPARγ) is produced. In the present context, it is to be understood that the active ingredient of said medicament may be the corresponding androstene (or androstane) derivative or an ester thereof with organic or inorganic acid. In the case of an organic acid, it can be comprised of up to 25 carbon atoms (S4 and S8). The effect of the medicament can be magnified or prolonged by simultaneously administrated sulphatase inhibitor, such as Coumate®.

c) Synthesis of 3β-sulfates S4 (R₁═SO₃H) and S8 (R₁═SO₃H) according to the method described by Arnostova, Libuse et al: Org. Chem. Biochem.,Czech. Acad. Sci., Prague, Czech. Synth, Commun. (1990), 20(10),1521-9.

The corresponding androstane derivative (S8, formula shown above) is produced from 17α- or 17β-AED (inversion of 17β- to 17α through Mitsunobu reaction, described in M&M) through protection of hydroxy groups with acetate and then reducing the double bound with Raney catalyst.

In an advantageous embodiment, said enzyme is DHEA-sulfotransferase or a phenolsul-photransferase. In one particular embodiment, the present medicament is a medicament, which enhances the effect of an estrogen receptor-a (ER-α) blockade.

The invention also includes the medicaments produced according to the method described above per se, as will be exemplified below.

The medicament produced according to the present method is useful for the treatment and/or prevention of a condition selected from the group consisting of urothelial cancers, gastric cancers, cancers of the smaller intestine, pancreatic cancers, tumours derived from endothelial cells, leiomyosarcomas, cancer of the colon, chorioncarcinomas, adenocarcinomas of the lung and liposarcomas, and pathology of the eye tissues, such as cells of the macula and glaucoma

In one embodiment, the invention relates to the use of 5-androstene-17α-ol-3β-sulfate (17α-AEDS) and/or androstane-17o-ol-30-sulfate in the manufacture of an immunomodulating medicament, e.g. for the treatment and/or prevention of an inflammatory disease, such as rheumatoid arthritis, arthrosis, or inflammatory bowel disease, or a disease caused by an exaggerated or persisting T-helper-1 response, such as multiple sclerosis or Guillain Barrés syndrome. The invention also relates to 5-androstene-17α-ol-3β-sulfate (17α-AEDS) and/or androstane-17α-ol-3β-sulfate for use as medicaments.

The present medicaments are suitable for administration in either their native form or in the form of a hydrolysable prodrug, i.e. an inactive form of the drug which is easily converted into active drug through hydrolysis in the environment of choice. Such a prodrug may be an ether or an ester of said medicament. Such a drug or prodrug is suitably administrated by topical injection, by intratumoural injection, by parenteral administration or intra-arterially, through selective Catheterization of an artery supporting a tumour or a site of pathologic neovascularisation, in the form of a pharmacologically acceptable sterile solution or suspension. A simultaneous injection of soluble starch particles of calibrated size (Spherex®) may enhance and prolong the effect of the drug and will also counteract the stimulus to neovascularisation and regrowth of the tumour produced by the resulting hypoxia.

The drug may be used as topical in the form of a pharmacologically acceptable solution, cream or jelly with for instance cyclodextrin applicated to the eye, to the mucosa of mouth, nose, vagina or rectum. On the skin a compress soaked in drug solution may be used for instance in preventing excessive scar-formation. The drug may also be taken orally, as rectal or vaginal suppositories, creams or enemas.

A 3β-sulfate according to S4 (R₁═SO₃H) or S8 (R₁═SC₃H) may also be taken per os in the form of capsules or in the form of tablets, mixed with a proton-pump inhibitor (to provide suitable pH) to reach a target in the gastric mucosa in the case of gastric cancer.

In malignancies of the smaller intestine or Crohns disease the drug may be taken as an entero-capsule, either in the form of suitable sulfated form or as native steroid (S4 or S8) as DHEA-sulphotransferase is present in the smaller intestine.

The sulfates of drugs S4 or S8 may be given intravesically to the urine bladder through a catheter a demure of a sterile solution in the case of superficial bladder cancer.

The compounds according to the invention, as defined by formulas (I) and (II) above, may be combined with cytotoxic drugs such as anthracyclines, such as doxorubicin, daunorubicin, epirubicin, idarubicin or mitoxantron, vincaalkaloids such as vinblastin, vincristin, vindesin or vinorelbin, taxanes such as docetaxel or paclitaxel, alkylating drugs such as ifosfamid, cyclofosfamid, busulfan, thiotepa, nitrosoureas such as lomustine, chlorambucil, dacarbazine, cisplatin, paraplatin or oxaliplatin, topoisomeraseII-lnhibitors such as etoposid or teniposid, topoisomerase-I-inhibitors such as topotecan or irinotecan, antimetabolltes such as methotrexate, mercaptopurin, cytarabin, 5-fluorouracil, gemcitabin, bleomycin, mitomycin, amsakrin, asparaginase, altretamine, hydroxycarbamide, miltefostin, estramustine, procarbazin or DTIC.

In one embodiment, the effect of the novel compounds according to the invention, as defined by formulas (I) and (II), may also be attenuated through the use of corticosterolds, retinolds, deltanoids, thyroid hormones, sex steroids and other nuclear receptor ligands.

### DETAILED DESCRIPTION OF THE DRAWINGS

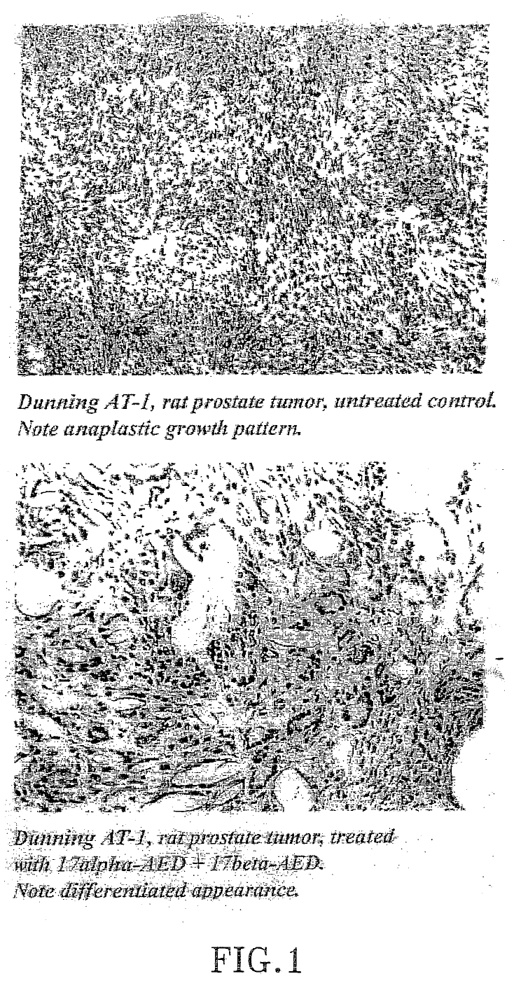

FIG. 1 is a picture of undifferentiated control and tumour treated with 17α17β-AED.

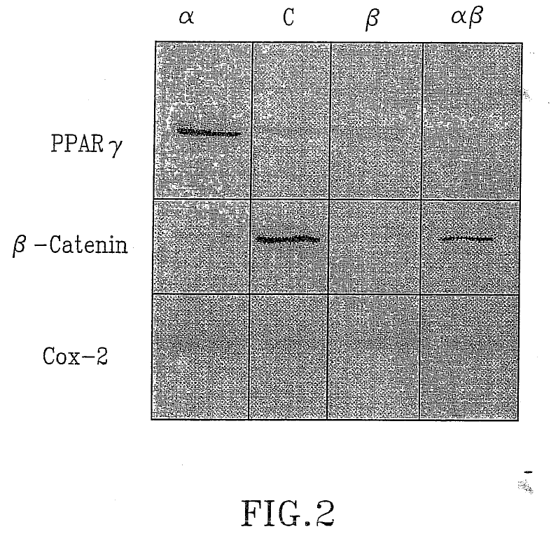

FIG. 2 shows Western blots as discussed in relation to Table IV.

α=rat tumour exposed to 17α-AED (which is represented to the left of control to point out the difference in exposure time compared to the other treatments). The drug was allowed to act for 96 hours.

β=tumour treated with 17β-AED, with an 8 times higher dose acting for 456 hours.

α+β shows sequential treatment with a for 96 hours followed by B for 360 hours.

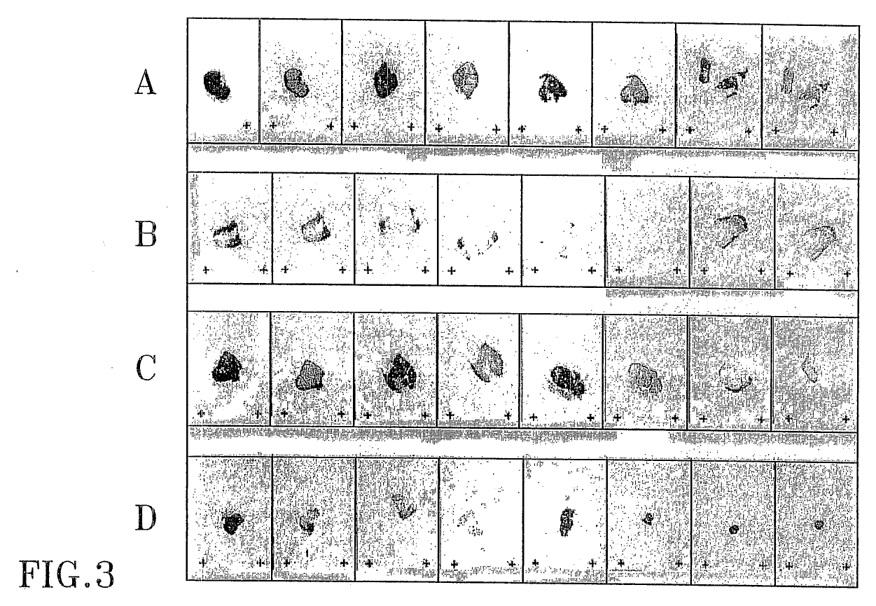

FIG. 3 illustrates effects on expression of VEGF of α-AED, β-AED, 5-androstene 3β,7β,17α-triol and 17αOH-pregnenolone. A: Untreated controls. Stained section to the left followed by negative control of same tumour. B: 17alpha-AED; Stained section to the left followed by negative control of same tumour. C: 17beta-AED; D: From left to right 1-3 tumour samples treated with androstene-3beta,7beta,17alpha-triolstained for VEGF, 4 negative control, same treatment,

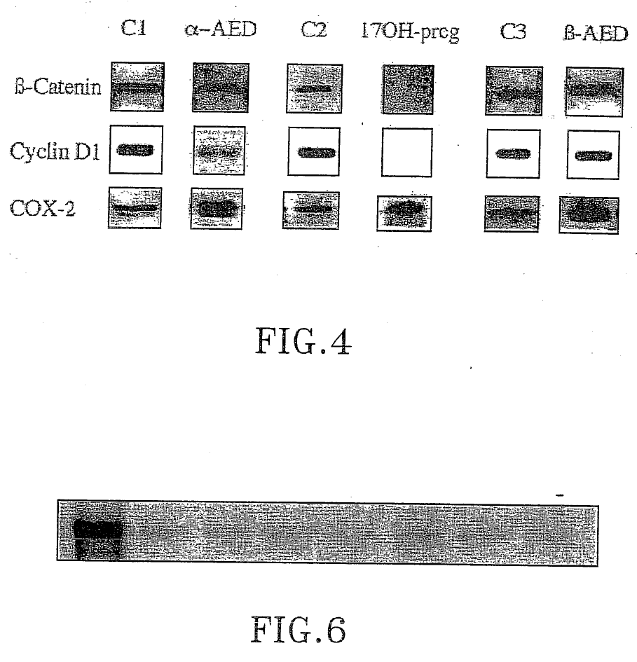

FIG. 4 shows protein blots of samples of Dunning AT-1 rat tumours treated with α-AED, 17αOH-pregnenolone and B-AED as well as untreated samples (C1-C3). Influence of treatment on expression of β-catenin, cyclin D1 and COX-2 is demonstrated by representative examples.

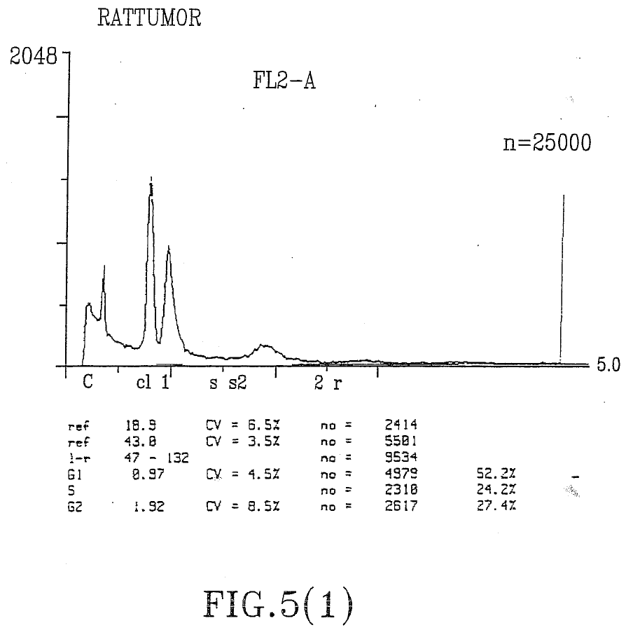

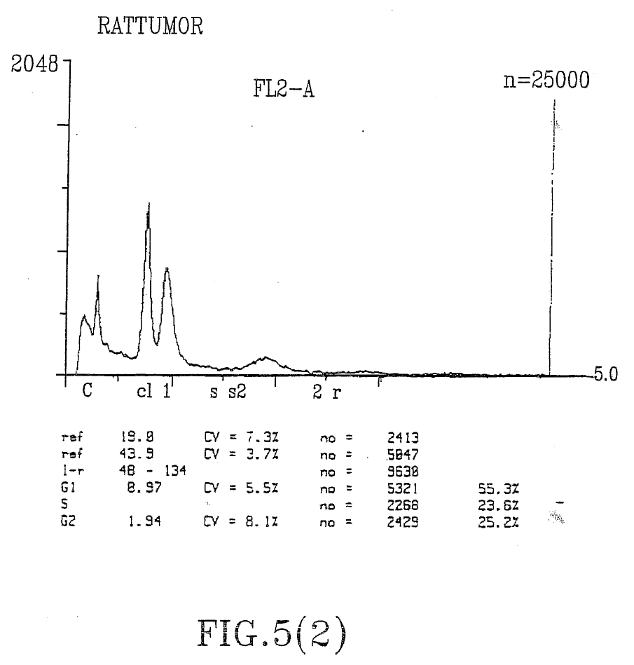

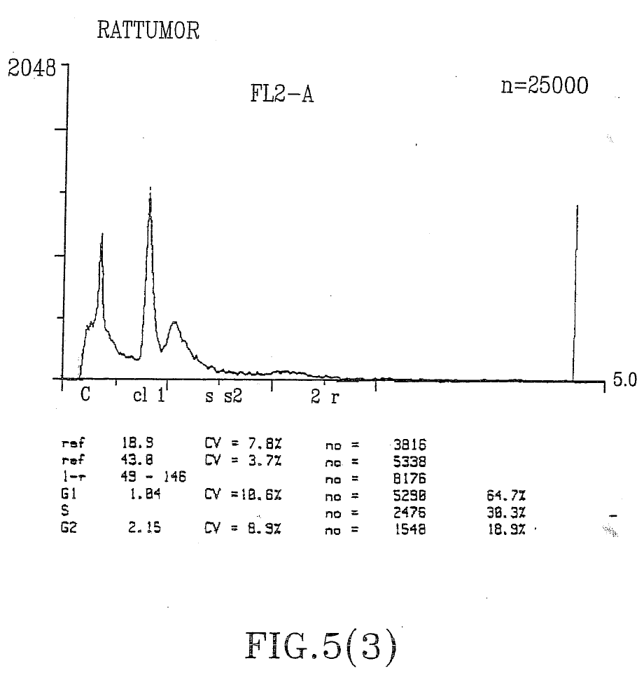

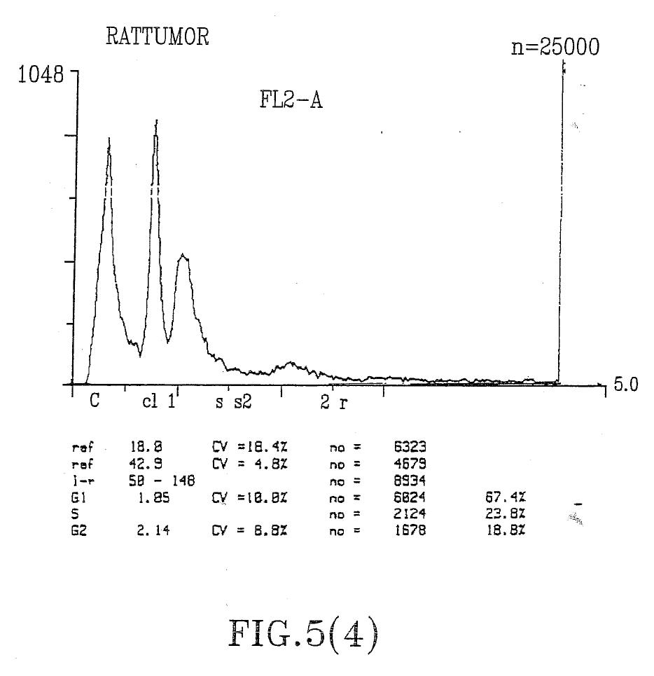

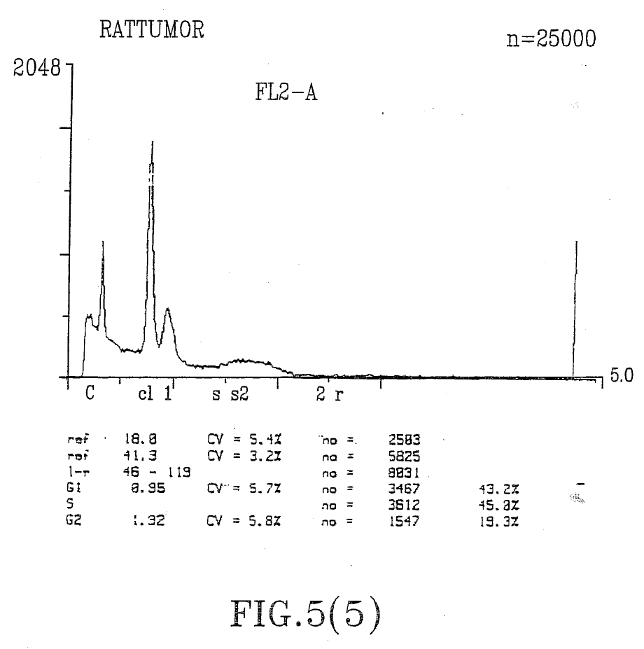

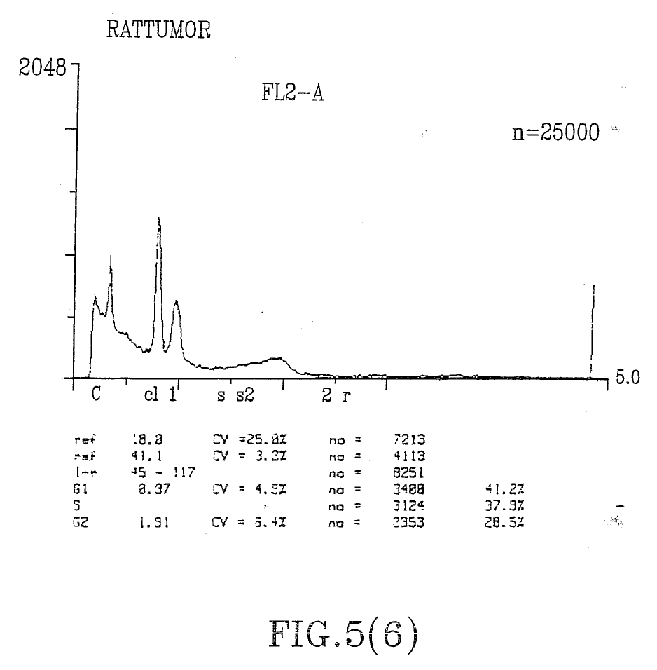

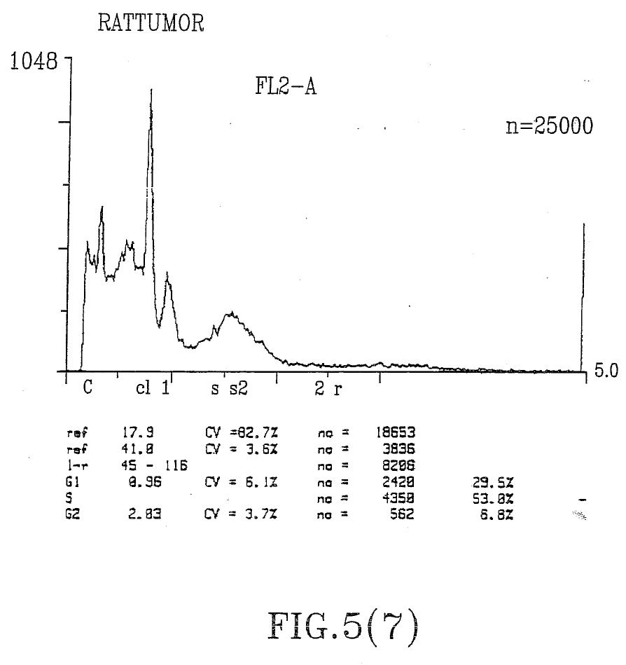

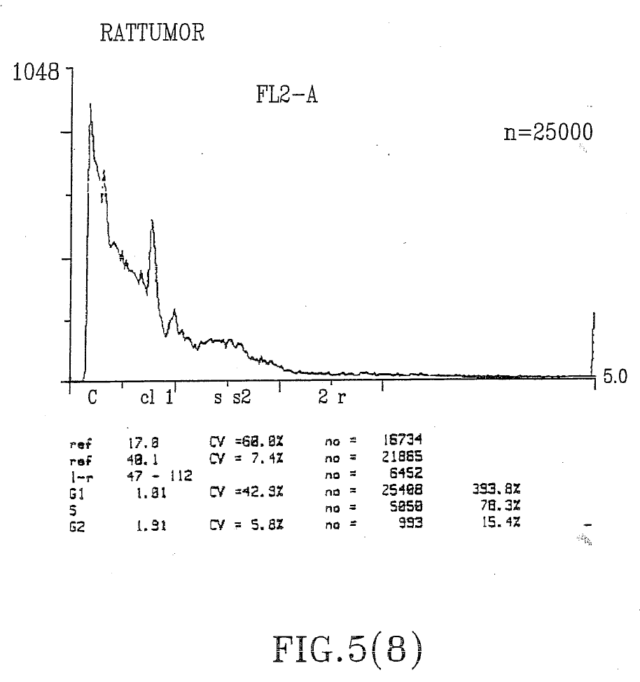

FIG. 5 shows representative examples of the effects on the cell cycle of Dunning AT-1, rat prostate cancer after treatment with 17α-AED (alpha A-D) compared to untreated control tumours (CA-CD). The untreated tumours show a strong expression of cyclin D1 in accordance with a large proportion of the cells in G1. In tumours treated with 17α-AED there is a decrease in G1 and an increase of cells in S-phase, paralleled by a decrease in cyclin D1. As there is no increase of cells in G2, the pattern combined with the results from protein blotting and demonstrated lack of apoptosis, indicates cell-death of non-apoptotic nature, in S or G2.

FIG. 6 illustrates how α-AED, 17OH-pregnenolone and 5-androstene-3β,7β,17α-triol and their corresponding 3β-sulfates were investigated for PPARγ-ligand activity in a ligand-induced coactivator assay. As positive control SRC-1 was used and as negative control rat liver cytosol from a male rat. The only sample demonstrating PPARγ-ligand activity is the 5-androstene-17α-ol-3β-sulfate.

### EXPERIMENTAL PART

### EXAMPLE 1

### Synthesis of 3β,7β,17β-trihydroxy-androst-5-ene

In this experiment, 3β,7β17α-trihydroxy-androst-5-ene (compound 6) was prepared as shown in Scheme 1 below, wherein (a) is NaBH₄, EtOH; (b) is Ac₂O, pyridine, DMAP; (c) is t-BuOOH, Cu (I)I, acetonitrile; (d) is NaBH₄, CeCl₃* 7H₂O, EtOH; and (e) is KOH, MeOH.

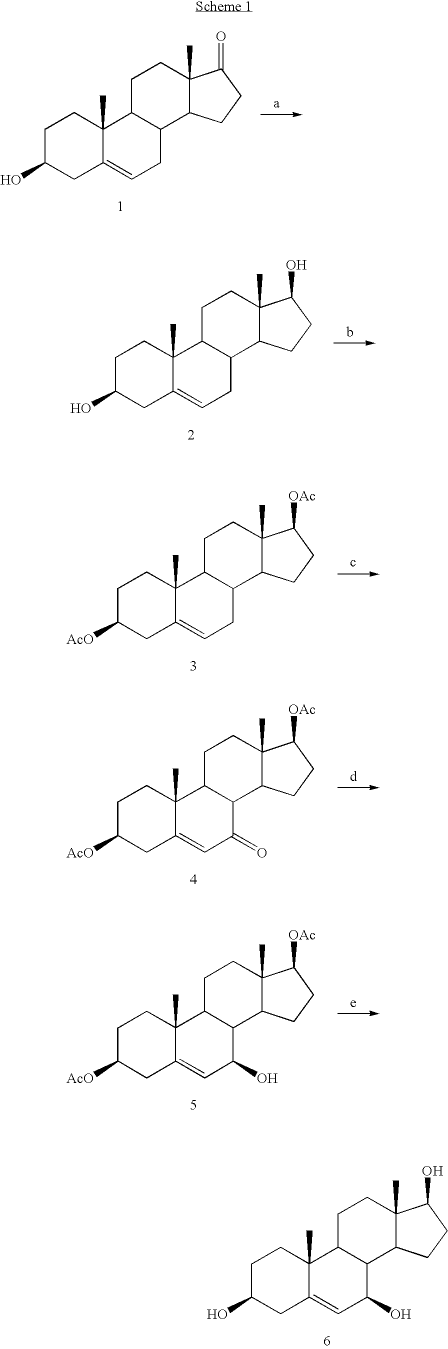

### Compound 2: 3β,17β-dihydroxy-androst-5-en

Compound 1 (1.00 g, 3.46 mmol) was dissolved in dry ethanol (15 ml) and NaBH₄ (196 mg, 5.20 mmol) was added slowly. After 1 hour at room temperature aqueous NaOH (2M, 6 ml) was added carefully and the mixture was extracted three times with diethyl ether. The combined extracts were dried over MgSO₄ and the solvent evaporated.

Yield: 91%.

¹H-NMR (CDCl₃, 400 MHz) 0.76 (s, 18-H), 1.05 (s, 19-H), 3.52 (m, 3α-H), 3.65 (t, 17α-H), 5.35 (d, 6-H)

### Compound 3: 3β,17β-diacetoxy-androst-5-en

Compound 2 (880 mg, 2.93 mmol), pyridine (25 ml), acetic anhydride (2 eq.) and DMAP (10 mol %, 29 mg) were heated at 100° C. for 2 hours, The reaction mixture was poured into water and the solid was filtered off, washed with diluted hydrochloric acid, NaHCO₃ and water and finally crystallized from ethanol.

Yield: 87%.

¹H-NMR (CDCl₃, 400 MHz) 2.03 (s, 17β-OAc), 2.05 (s, 3β-OAC), 5.38 (d, 6-H)

### Compound 4: 3β,17β-diacetoxy-androst-5-en-7-one (Salvador, J. A. R.: Melo, M. L. S.: Neves, A. C. S. Tetrahedron Lett. 1997. 119-122)

To a solution of Compound 3 (650 mg, 1.80 mmol) in acetonitrile (12 ml) under argon, copper (I)-iodide (3.6 mg, 0.18 mmol) and t-butyl-hydrogenperoxide (2.5 ml, 10 mmol) were added. After 24 hours under magnetic stirring at 55° C., the solution was poured into Na₂SO₃-solution (100% aqueous) and extracted with diethylether. The extract was washed with aqueous saturated solution of NaHCO₃, brine and water, dried over MgSO₄ and the solvent evaporated.

Yield: 45 %.

¹H-NMR (CDCl₃, 400 MHz) 5.72 (d, 6-H).

### Compound 5: 3β,17β-diacetoxy,7β-hydroxy-androst-5-en

The reduction procedure of Luche (Luche, J.-L. J.Am.Chem.Soc. 1978, 100, 2226) was followed: A solution of compound 4 (330 mg, 0.86 mmol) in dry ethanol (10 ml) was cooled with stirring to −78° C. CeCl₃* 7H₂C (335 mg, 0.86 mmol) was added followed by NaBH₄ (48 mg, 1.29 mmol).

The reaction mixture was warmed slowly to 0° C. and stirred for 30 minutes. Aqueous NaOH (2M, 6 ml) was added carefully and the mixture extracted three times with diethylether. The combined extracts were dried over MgSO₄ and the solvent evaporated. Compound 5 (150 mg, 0.40 mmol) so produced was saponified by refluxing for one hour in 5% potassium hydroxide in methanol. After working up the mixture, the product was recrystallised from acetone/water. Yield: 80%/.

¹H-NMR (MeOd, 400 MHz) 3.40 (m, 3α-H), 3.57 (t, 17α-H), 3.85 (d, 7α-H), 5.21 (s, 6H)

### EXAMPLE 2

### Synthesis of 3β,7β,17α-trihydroxy-androst-5-ene

In this experiment, 3β,7β17α-trihydroxy-androst-5-ene (compound 12) was prepared as shown in Scheme 2 below, wherein (a) is t-butyidimethylsilyl chloride, Imidazole, DMF; (b) is NaBH₄, CeCl₃* 7H₂O, EtOH, (c) is p-nitro-benzoicacid, PPh₃, DEAD, toluene; (d) is tBuOOH, Cu (I)I, acetonitrile; and (f) is n-Bu₄NF, THF, KOH, MeOH.

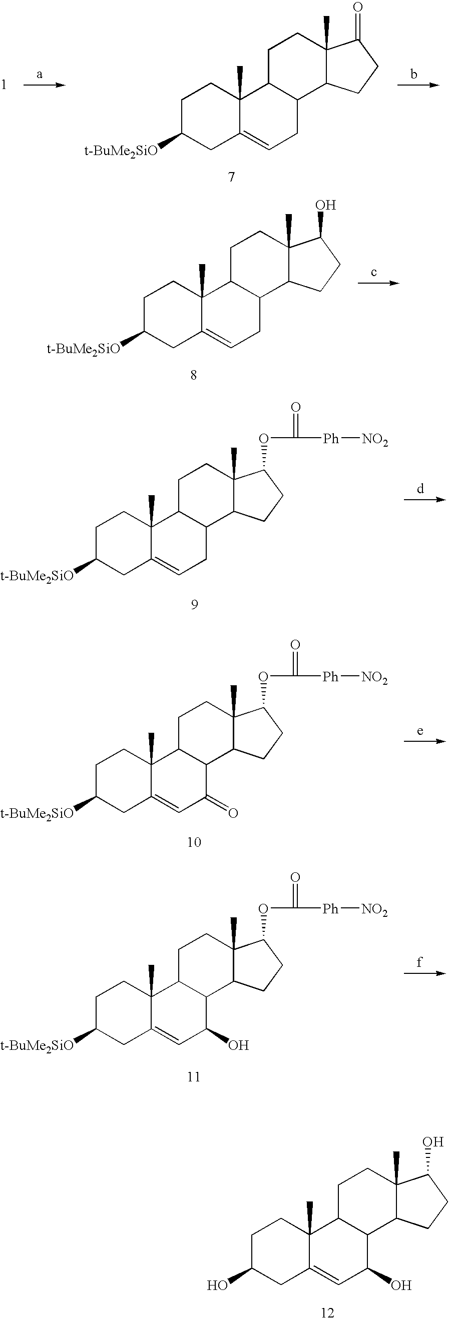

### Compound 7: 3β-(Dimethyl-t-butylsiloxy) androst-5-en-17-one (Mitsunobu reaction)

A solution of compound 1 (2.88 g, 10 mmol), imidazole (1.7 g, 25mmol) and t-butyidimethylsilyl chloride (1.8 g, 12 mmol) in dry DMF (20 ml) was kept under argon over night, then poured into water extracted by chloroform and the solvent evaporated. Yield: 980/0.

¹H-NMR (CDCl₃, 400 MHz) 0.06 (s, Me₂Si), 0.88 (s, 18-H), 0.89 (s, tBu), 1.02 (s, 19-H), 2.41 (dd, 16-H), 3.50 (m, 3α-H), 5.37 (d, 6-H)

### Compound 8: 3β-(Dimethyl-t-butylsiloxy),17β-hydroxy-androst-5-en

Yield: 92%.

¹H-NMR (CDCl₃, 400 MHz) 3.64 (t, 17α-H)

### Compound 9: 3β-(Dimethyl-t-butylsiloxy)17α-benzoyloxy-androst-5-en

Compound 8 (1.7 g, 4.19 mmol), p-nitro-benzoicacid (1.8 g, 11 mmol) and triphenyl-phosphine (2.8 g, 10.5 mmol) were solved in toluene (25 ml) under argon and heated to 30° C. DEAD (1.53 ml, 10 mmol) was added slowly. The mixture was refluxed for 2 hours, the solvent evaporated and the solid residue chromatographed by pentane/diethyether=95/5.

Yield: 68%.

¹H-NMR (CDCl₃, 400 MHz) 5.2 (d, 17β-H), 8.2-8.35 (Ar)

### Compound 10: 3β-(Dimethyl-t-butylsiloxy),17α-benzoyloxy-androst-5-en-7-one

Yield: 36%.

¹H-NMR (CDCl₃, 400 MHz) 5.68 (d, 6-H)

### Compound 12: 3β,7β,17α-trihydroxy-androst-5-en

¹H-NMR (CDCl₃, 400 MHz) 3.57 (m, 3α-H), 3.75 (17α-H), 3.80 (d, 7α-H)

### EXAMPLE 3

### Evaluation of Effects

Material and Methods

Animals 1

Viable tumour pieces of Dunning R 3327, AT-1, rat prostatic tumour, previously grown on Copenhagen-Fischer rats previously treated as follows were taken for investigation:

Group 1 a single dose of 80 mg Δ5-androstene-3β,17β-diol (Sigma Chemicals) s.c.

Group 2 and 3 a single dose of 10 mg of Δ5-androstene-3β,17α-diol, (Steraloid Inc.).

Group 4 served as untreated, tumour-bearing controls. In all treatments equal amounts of PEG 400 (Sigma Chemicals) and ethanol, 0,5 ml was used as vehicle and injected s.c. adjacent to the tumour site.

After 96 hours group 3, previously treated with 10 mg of Δ5-androstene-3β,17α-diol, received a single injection of 80 mg of ΔS-androstene-3β,17β-diol in the same way as group1.

In group 2 the experiment was terminated after 96 hours and for the remaining groups after 19 days through asphyxiation of rats with carbon dioxide. The local animal ethics committee had accepted the experiment. Further details of this experiment are as described in (Antitumoural activity of 17α-AED and 17-β epimers in vivo, in Dunning AT-1 prostate cancer in rat: Hagström et al. unpubi.).

Immunostaining

Immunostaining was done on formalin fixed sections against VEGF, clone C-1, IgG2a, sc-7269, Santa Cruz.Worklng dilution 1:100. As negative control antibody a mouse IgG, clone DAK-GO1, kappa, x 931, Dako, was used with a working dilution of 1:50.

Tumours were investigated for increased apoptosis with the Apop-Tag in situ apoptosis detection kit (Oncor, Gaithersburg, Md.).

Animals 2

The animal experiment described above was repeated.

Group 1 received a single dose of 10 mg 17α-AED sc. near tumour.

Group 2 received a single dose of 80 mg 17β-AED in the same way.

Group 3 received a single dose of 25 mg 17α OH-pregnenolone in the same way.

Group 4 received a single dose of 7,5 mg 5-androstene-3β,7β,17α-triol.

A fifth group served as tumour bearing controls.

Cell Lines

Human prostatic cancer cell lines, PC-3 and DU-145, were used. Frozen cell lines were established in culture. DU-145, which was received as a tumour piece, was first disintegrated in a sterile Petri dish, using a pair of scissors. The disintegrated tumour was transferred to a 75 cm³ cell-culture flask containing medium. For medium, Ham's F 10 was used for PC-3 and RPMI 1640 for DU-145.

FBS,10%, 0.2% NaHCO3 (7.5%), 2 mM L-glutamine (200 mM) and 0.005 mg/ml gentamycin (5 mg/ml) were added to culturing media for both cell lines.

Cells were grown in 75 cm³ cell culture flasks until nearly confluent and culture medium was changed twice weekly. A solution of 17α-AED, 17-β-AED as well as a mixture of both in equal parts of DMSO and ethanol diluted in culture medium, not allowing the concentration of DMSO in culture-flasks to exceed 0,15%, was added respectively.

The concentration of AED in each culture flask was 100 nM. Controls were exposed to the same concentration of DMSO and ethanol in culture medium.

Exposure was maintained for 96 hours and cells in culture flasks were then treated with 5 ml of trypsin (0.25%) for PC-3 and trypsin and 3 ml of Versene (0.2 mM) for DU-145. Cells were then mechanically loosened, using a glass spatula. 100 μL aliquots were removed for Trypan blue exclusion trial. 200 μL aliquots were removed for cytospin preparations. The remaining 700 μL samples were then centrifuged for 5 minutes at 510×g at 4° C. Supernatant was removed and cells were resuspended in medium in 1 ml of insect cell lysis buffer (Pharmingen #21425A) and incubated for 30 minutes on ice. The cell lysates were then stored at −70° C. until analysis.

Apoptosis Assay

Aliquots of a 100 μL of cell lysate were removed for the caspase assay. Each portion was mixed with 1 ml protease assay buffer and Ac-DEVD-AMC substrate (Pharmingen #66081U) with a final concentration of 20 μM. Solutions were incubated for 60 minutes at 37° C.

To determine background fluorescence a control was prepared, containing no cell lysate but the same amounts of protease assay buffer, lysis buffer and Ac-DEVD-AMC substrate.

Fluorescence was measured using a spectrofluorometer (RF 540, Shimadzu Data Recorder DR3, instrument AB, Lambda) at 415-450 nm with an excitation wavelength of 380 nm.

Trypan Blue Exclusion Trial

100 μL aliquots of the initial cell suspension in PBS were used to determine the viability of cells collected by means of the trypan blue exclusion assay. 10 μL of the cell suspensions were mixed with 10 μL trypan blue solution (Sigma Chemical Co. # T8154) and left to sit for 3 minutes. The numbers of viable and dead cells were counted in a hemocytometer.

Photoregistration

In order to discover a possible growth inhibitory action cultures containing 17α-AED as well as controls containing a similar number of cells were grown in Petri dishes with a grid, making an identification of a special area possible. Cultures were photographed every 24 hours until confluence.

Effects of HER-2 and 17α-AED-3β-sulfate in breast and orostate cancer cell lines. Cell cultures of prostate cancer cell lines PC-3 and DU-145 as well as mammary cancer cell lines MCF-7 and SKBBR-3 were grown in flasks as previously described in the presence or absence of a 200 nM 17α-AED. Cultures containing 17α-AED were further subdivided and these cultures were grown in the presence or absence of Herceptin®, her-2 antibodies, using a stem solution of 1 mg/ml which was further diluted, making the final concentration 1:150 in the cell suspension. As no preservative was added to her-2 antibodies, they were added at 2 occasions, 24 hours apart during 72 hours of incubation, (taking the limited half-life of the antibody into consideration).

Representations of each cell line containing no steroid were also treated with herceptin as above. For each cell line and variation of treatment 3 different culture flasks were used.

Further, a mixture of 100 μL rat liver cytosol and 20 μL of 3′-phosphoadenosine-5′-phosphosuiphate (PAPS), corresponding to approximately 0,16 mg of PAPS was shared between and added to cultures containing 17α-AED.

Finally, for each cell line three samples were kept as untreated controls. To estimate the number of viable cells a solution of chlorofluoresceindiacetate was added to the cultures on the last day of culture. This chemical penetrates freely into cells, but is metabolized in the living cell into a form with green fluorescence unable to leave the cell. As counterstalning, to detect dead cells, propidiumiodide, giving a red fluorescence, was added 15 minutes before analyzing the samples in a Facscan. Time for 10000 cells to pass the detector in the Facscan was estimated in order to detect a possible growth inhibitory effect, not resulting in an increased number of dead cells.

Combined Influence of Estrogen-Receptor Blockade 17α-AED-3β-Sulfate and HER-2 Antibodies in DU-145 and PC-3 Cell Cultures

Based on previous experiment cell culture experiment was repeated with the two prostate cancer cell lines. According to literature, estrogen receptor β is expressed in both cell lines and estrogen receptor x in PC-3 cells only. To block estrogen receptors, ICI 172,780 in a 50 nm concentration was used and added at two occasions 36 hours apart. The estrogen receptor blocked cultures were then treated with ±HER2 antibodies and ±17α-AED-3β-sulfate in the concentrations and manner as in experiment with PC-3 and DU145 with the exception that estrogen receptors were blocked as described.

Cell-Culture of 3T3-L1 Fibroblasts

3T3L1 fibroblasts from ATCC (embryonic muscle cell-line) batch F-12732 (batch-date 930301) were grown in 500 ml of DMEM, 50 ml of fetal bovine serum (FBS) and 11 ml of PEST. Medium was changed every second day. Cells were passaged when they were 80% confluent.

For differentiating purpose fibroblasts were incubated in DMEM+10% FCS+5 μg/ml of insulin. 0.1 mM of IBMX and 0.25 μM of dexametason was added. After 2 days cells were transferred to DMEM+10% FCS+5 μg/ml insulin. Medium was then changed every second day and consisted of DMEM+10% FCS. Ethanol in distilled water was added; keeping the ethanol concentration below 0.1%. This served as positive control for differentiation. Fibroblasts were also incubated with the same solutions except that IBMX and dexa-methasone were exchanged with solutions of 17α-AED in ethanol and water, making the concentration of 17α-AED 100 or 200 nM in the cultures. Cultures were kept in incubator until confluent.

Protein Blotting

Thawed rat tumours from both experiments above were homogenized in ice cold lysis-buffer. (160 mM NaCl, 10 mM HEPES, 2 mM CaCl2, 5% SDS, 0,5% Triton X-100, 100 μg/ml phenyl-metylsulfonyl fluoride, 1 μg/ml leupeptin and 2 μg/mi aprotinin), placed on Ice for 15 minutes and following clarification centrifuged for 10 minutes at 13,000 g. Protein content of homogenates were determined by Lowry assay.

Protein in lysates (90 μg) were separated by electrophoresis using 8% SDS-PAGE and separated proteins were transferred onto a nitrocellulose membrane (Amersham) in trans-blot electrophoretic transfer cell (Bio-Rad Laboratories).

After trans-blotting and blocking with non-fat milk in TTBS (1×TTBS: 20 mM Tris, 150 mM NaCl, pH 7.5, 0,1% Tween-20) blots were probed with mouse monoclonal IgG1 PPARγ anti-body (Anti-PPAR-gamma (ES) Santa Cruz Biotechnology) diluted 1:400 in TTBS containing 3% (w/v) non-fat dried milk.

After washing in TTBS the blots were reincubated with horseradish peroxidase conjugated secondary antibody (anti-goat antibodies 2020, Santa Cruz) diluted 1:4000 in TTBS containing 3% (w/v non-fat, dried milk for one hour. Blots were developed using an enhanced chemiluminescence system (ECL, Amersham) and exposed to HyperFilm ECL (Amersham). Optical density was measured densitometrically.

The blots were striped and rehybridized with antibodies against β-catenin (C-18, sc. 1496) and COX-2 (C-20, sc 1745) antibodies.

All-experiments were repeated twice in separate assays.

In the second animal experiment tumours were investigated for expression of β-catenin, COX-2 and cyclin D1 (A-12, sc. 8396).

Protein blotting was repeated for DU-145 and PC-3 cell lines, which were also investigated for expression of PPAR5 through antibody (H-74, sc 7197).

Preoaration of (³⁵S) Methionine Labeled SRC-1

ApcDNA3-SRC-1 construct was used as template to prepare (³⁵S-methionine)-SRC-1 by in vitro transcription and translation using the TNT® Coupled Peticulocyte Lysate System (Pro-mega).

Ligand-induced interactions between GST-PPARγ and SRC-1: To generate GST-PPARγ fusion protein, mouse PPARγcDNA was inserted into the NcoI-HindIII sites of pGEX-KG, and protein was then expressed in E.coll, strain Y 1090. The bacteria were lysed by sonication in TEDG buffer (50 mM Tris (pH 7.4), 1.5 mM EDTA, 10% glycerol, 0.4 M NaCl, 0.1 mM DTT) containing the following protease inhibitors: 0.5 mM PMSF, 1 mM benzamidine, 10 μg/ml leupeptin, 10 μg/ml antipain and 10 μg/ml aprotinin. The lysates were centrifuged at 100,000×g for 60 min in a SW 41 rotor using a Beckman L8-70 M ultracentrifuge. The fusion protein was immobilized on GSH Sepharose beads (Pharmacia) and then incubated with potential ligands in (FIG. 5) 10 mM Tris (pH7.4), 0.12 M KCl, 8% glycerol, 4 mM DTT, and 0.5% CHAPS (buffer AA) for 30 min at room temperature. Thereafter, (355 methionine) SRC-1 (ca 0.1 μCi) was added, and the beads were incubated for another hour at 4° C. and then washed extensively with buffer A. SDS sample buffer was added to the beads, and the samples boiled prior to separation on 7.5% SDS-PAGE. Radioactivity was detected by autoradiography.

Ligand-Induced Coactivator Interaction Assay

α- and β-AED were investigated in above assay for PPARγ ligand activity in 10 and 100 μM solutions in ethanol 10%, in distilled water, 10 μL aliquots of α-AED-solution or β-AED-solution were also added to a preparation of a male rat liver cytosolic preparation prepared as follows:

A homogenate of a rat liver, a known source of steroid sulphotransferase activity, was prepared using a food processor. The liver homogenate was protected from serine-proteases by the addition of 2 mmol of PMSF, 1 mmol of EDTA and 10 mmol Tris-HCl buffered to a pH of 7,40. The homogenate was prepared at 4° C. and then centrifuged at 15000 g for 10 minutes, followed by centrifugation at 100000 g for an hour at 4° C. The supernatant was divided into test tubes containing one of the following steroids: 17β-ASD or 17α-AED in 5 μM concentration together with 100 μL of liver cytosol. One test tube served as positive control and contained SRC-1.

In a second experiment the following steroids were tested for activity: 17α-AED, 17OH-pregnenolone and 5-androstene-3β,7β,17α-triol with and without 10 μL of 5′-phospho-adenosine-3′-phosphosulphate (PAPS), Sigma Chemicals, corresponding to 0,1 mg of PAPS. One test tube served as a positive control and contained SRC-1 instead of steroid. One test tube contained liver cytosol where the steroid sulphotransferase was inactivated by heating to 45° C. for 30 minutes. Tubes were stirred at 20° C. for 1 hour and the contents were investigated in the receptor-coactivator assay for ligand activity (FIG. 5).

Synthesizing 3β-Sulfate of 17α-AED, Identification of Molecular Structure and Verification of Activity in Ligand-Induced Coactivator Interaction Assay 1

17α-AED was treated in the method described by Arnostova, Libuse M.; Pouzar, Vladimir; Drasar, Pavel. Inst. Org. Chem. Biochem., Czech. Acad. Sci., Prague, Czech. Synth. Commun. (1990), 20(10), 1521-9, where acetate is used as protection group and pyridine —SO₃ complex as the sulfating agent. Deprotection with an excess of 0,8M NaOH in MeOH-water provides the desired hydroxy sulfate, which is investigated, for PPARγ-activity in the ligandactivated-coactivator receptor assay described above. This shows PPARγ-activity for the 17α-AED-3βsulfate.

Results

Morpholoy

Sequential treatment with first 17β-AED followed by 17β-AED in Dunning AT-1, rat prostatic tumour changed tumour appearance from anaplastic pattern to a tumour showing glandular differentiation. As human prostatic carcinomas express PPARγ and since activation of this receptor can lead to differentiation in other types of tissue expressing PPARγ, protein blotting with antibodies against PPARγ was performed in order to see if an attenuation of the expression of this nuclear receptor could explain the differentiation.

3T3-L1 Mouse Fibroblasts

A pre-confluent culture of 3T3L1 mouse fibroblast culture was exposed to 100 and 200 nmolar 17α- or β-AED to see if a differentiation into adipocytic phenotype would take place. Positive control containing IBMX and dexamethason differentiated as expected after reaching confluence.

No differentiation was observed for the androstenediols but a marked inhibition of cell proliferation was seen in the fibroblast culture exposed to a 200 nM concentration of 17α-AED.

Apoptosis, Cell Viability and Growth Inhibition

Investigation for increased apoptosis in sections of rat prostate tumour treated with 17α-AED with TUNEL technique showed no sign of increased programmed cell death compared to control (Hagstöm et al. Antitumoural activity of 17α-AED and 17-β epimers in vivo, in Dunning AT-1 prostate cancer in rat).

Measurement of changes in caspase- 3 or caspase-7 (DU-145 lacks caspase 3 activity during apoptosis. Instead it has caspase-7 which is less effective in mediating apoptosis than caspase-3. Both caspases, however, recognize the same amino acid motif and the substrate used is thus suitable for detecting both) showed a significant decline when DU-145 was grown in the presence of 17α-AED compared to growth in 17β-AED or controls. Interestingly a significant decrease in total number of cells was noted after treatment with 17α-AED in DU-145 cells. This suggests a growth inhibitory effect of both steroids in DU-145 cells. Measurements of fluorescence were corrected for differences in cell number, but the decrease in fluorescence in 17α-AED treated samples compared to controls or other steroids remains. This suggests a protective effect against apoptosis from 17α-AED in DU-145 as well as PC-3.

Caspase 3-mediated fluorescence in PC-3 cells treated with 17α-AED shows an essentially unaltered intensity compared to untreated controls. When intensity is corrected for cell numbers there is however a decrease, corresponding to a decrease in apoptosis, which is probably significant. For 17β-AED approximately a doubling of fluorescence, corresponding to an increase in apoptosis was seen. Looking at total number of cells in the PC-3 culturesreveals an increase in total number of cells with 59% in cultures treated with 17α-AED compared to controls.

A smaller increase in cell number, 29%, was seen in samples treated with 17β-AED. A small decrease in proportion of viable cells is seen in PC-3 cultures treated with 17α-AED or 17β-AED. This is however outweighed by the increased number of total cells in these cultures, making the number of viable cells in these cultures equal the total number of cells in the cultures.

Accordingly, this experiment demonstrates that 17α-AED gives significant protection against apoptosis in two common examples of androgen refractory human prostate cancer; the cell lines PC-3 or DU-145. Further, 17β-AED increases apoptosis in PC-3 cells but not in DU-145.

Contrary to observations in breast cancer cell lines of earlier date by Loria, the combination of both steroids gave no significant effects on apoptosis in prostate cancer. 17α-AED as well as 17β-AED increases cell death in PC-3 cells, but also increases the number of viable cells.

Below, table I shows PC-3 and DU-145 cells treated with 17α-AED,17β-AED or both and number of dead and viable cells compared to untreated controls. Estimations for each group are based on two different measurements from three different culture flasks.

TABLE I

| | PC-3 | DU-145 | PC-3 | DU-145 | PC-3 | | | |
|---|---|---|---|---|---|---|---|---|
| | mean | mean | mean | mean | mean | DU-145 | PC-3 | DU-145 |
| | Viable | Viable | Dead | Dead | Tot. | mean | mean | mean |
| Group | cells | cells | cells | cells | cells | Tot. cells | % viable | % viable |
| α | 58(49-77) | 102(95-111) | 23(16-32) | 29(21-37) | 81(64-98) | 131(122-148) | 71.6 | 78.1 |
| β | 49(46-53) | 90(78-107) | 17(16-18) | 35(31-42) | 66(63-71) | 125(109-149) | 73.7 | 72.1 |
| αβ | 49(46-53) | 104(62-140) | 11(11-12) | 29(22-37) | 60(53-68) | −(84-176) | 80.0 | 77.9 |
| Control 1 | 48(44-51) | 146(137-161) | 9(9-10) | 47(41-52) | 57(53-60) | 193(181-212) | 84.0 | 75.5 |
| Control 2 | 37(22-41) | 129(116-152) | 8(5-10) | 43(42-45) | 45(32-56) | 172(158-193) | 80.2 | 75.1 |

Further, table II below shows the mean fluorescence in DU-145 and PC-3 samples estimated with spectrofluorometry in caspase-3, apoptosis assay,

TABLE II

| | Mean fluorescence | Mean fluorescence | Mean fluorescence | Mean fluorescence |
|---|---|---|---|---|
| | DU-145 samples | DU-145 samples/100 | PC-3 samples | PC-3 samples/100 |
| Groups | n = 3 | cells n = 3 | n = 3 | cells n = 3 |
| Fluorescence | 12.0 | | 25.2 | |
| control | | | | |
| α | 18.4(13.2-25.2) | 14.2((10.9-20.3) | 15.2(13.6-16.6 | 19.5(15.7-25.9) |
| β | 42.9(32.0-58.8) | 35.1(25.6-50.3) | 29.6(17.4-48.1) | 45.5(24.5-75.2) |
| αβ | 53.4(47.6-64.8) | 42.8(34.2-57.5) | 23.7(13.3-29.1) | 40.0(22.4-55.4) |
| Control 1 | 64.3(62.0-66.0) | 33.5(30.6-35.6) | 16.4(13.3-22.0) | 28.5(22.7-36.7) |
| Control 2 | 56.9(49.6-60.8) | 33.2(31.2-36.8) | 15.7(11.9-19.8) | 35.9(27.5-43.0) |

Table III below shows estimations of viability and apoptosis in HER-2/neu expressing DU-145 and PC-3 androgen-refractory human prostate cancer cell lines and human mammary cancer cell lines MCF-7 (estrogen receptor positive) and SKBR-3 (estrogen receptor negative) and the influence of HER-2/neu antibody herceptin, 3β-sulfate of 17α-AED and their combination.

TABLE III

Cell line

| | PC-3 | DU-145 | MCF-7 | SKBR-3 |
|---|---|---|---|---|
| Control: viable | 72% | 72% | 75% | 72% |
| Control: dead | 19% | 16% | 13% | 16% |
| α SO4: viable | 81% | 70% | 75% | 66% |
| α SO4 dead | 12% | 18% | 15% | 21% |
| Her-ab viable | 76% | 70% | 74% | 69% |
| Her-ab dead | 17% | 17% | 12% | 19% |
| α SO4+ Her-ab | 76% | 66% | 77% | 83% |
| viable | | | | |
| α SO4+ Her-ab | 16% | 18% | 13% | 7% |
| dead | | | | |

Measurements of fluorescent cells in Facscan were interpreted in the following way: Two major cell populations were found. One staining strongly for chlorofluorescelndiacetate (CFA) and very weakly for propidiumiodide (PI) counted as viable and the other with cells with opposite staining pattern, counted as dead.

A small cell population, staining strong for both, could contain early apoptosis.

The results showed similar figures for the proportion between cells estimated as viable or dead in the controls of all cell lines. 17α-AED-3β sulfate showed effects, though opposite in PC-3 and SKBR-3. The percentage of viable cells was increased and dead cells decreased in PC-3 cultures.

In SKBR-3 a decrease in viable cells and an increase in dead cells is seen in 17α-AED-3βsulfate treated cultures. Treatment of cell cultures with HER-2 antibodies shows no significant effects on the proportion between surviving and dead cells. The combination of HER-2 antibodies and 17(x-AED-3βsulfate seems to protect SKBR-3 cells where a significantly increased proportion of cells survives. There is a weak but similar tendency in the other cell types as well, except for DU-145 where this combination gives a reduction in surviving cells.

Accordingly, this experiment does not support the theory that HER-2 expression is the reason behind lack of apoptotic effects from 17α-AED-3βsulfate in these cell lines, except for perhaps DU-145. The result seen in 17α-AED-3βsulfate treated SKBR-3, shows that it is necessary to block estrogen to get an effect by 17═-AED-3βsulfate.

Blocking of Estrogen-Receptor in Human Androcen-Refractory Cancer Cell Lines PC-3 and DU-145

Results of estrogen-blockade with ICI 172,780 shows in PC-3 cells a considerable decrease in viable cells as well as an increase in cells staining weakly for both markers, DCFA and PI, the latter suggesting apoptotic cells.

In DU-145 result was less clear with no obvious increase in number of dead cells. Combined treatment with 17α-AED-3βsulfate and ICI 172,480 in PC-3 cells resulted in approximately 80% cell death. No increase in cell death was seen in DU-145 cells. The number of cells staining strongly for PI was decreased, especially in cells receiving a combined treatment.

Accordingly, this experiment Illustrates how an increased cell death is observed after blocking of estrogen-receptors in PC-3 cultures. A synergistic effect is observed when 17α-AED-3βsulfate is added. No obvious effect is observed in DU-145 cells.

Western Blotting Cell Lines

Western blotting as well as northern blotting of PC-3 and DU-145 cell lines treated with 17α-AED as well as untreated controls (3 samples of each) showed a weak expression of COX-2 and β-catenin in PC-3 and a much stronger expression in DU-145. Expression of PPARγ was much stronger in PC-3 than in DU-145. PPARδ is strongly expressed in DU-145, but only weakly in PC-3. Treatment with androstenediols separately or in combination showed no modulatory effect on the expression of PPARγ, PPARδ, COX-2 or β-catenin, whereas AEDS increased the expression of PPARγ significantly in both cell lines, AEDS, however, had no effect on the expression of PPARδ.

Western Blotting Cell Lines Treated with 17α-AEDS

The experiment is repeated in the same cell lines as above. 100 nM solution of 17α-AEDS is used instead of 17α-AED. In PC-3 cells PPARγ expression is increased. Expression of PPARγ was observed in DU-145.

Western Blotting Animals I

Western blotting was performed using samples from three different tumours treated with 17α-AED, 17β-AED and a sequential treatment with both. Antibodies against PPARγ, COX-2 and β-catenin were used. The experiment was duplicated, using a different control tumour, with identical result.

The numerical value of controls was set to 100. Mean values and range and p-values in table IV below.

TABLE IV

| | α-AED | β-AED | α + β-AED |
|---|---|---|---|
| PPARγ | 190(176-210) | 47(40-53) | 20(18-23) |
| β-catenin | 20(15-30) | 14(10-22) | 50(42-62) |
| COX-2 | 219(192-234) | 135(133-137) | NE(80, 80, 244) |

The unexpected finding of a doubled expression of PPARγ and the same time a very marked decrease in expression of β-catenin, while at the same time a doubling of COX-2 in tumour treated with α-AED is contradictory to what one would expect from a PPARγ-ligand (Also the apparently opposite effects from combining α and β-AED. As time of exposure to drugs is very different between the treatment arms, one must be very cautious in comparing data; between different treatments.

Western Blotting Animals II

Controls showed a high expression of β-catenin, cyclin D1 and COX-2. In samples treated with α-AED there was a marked decrease in the expression of β-catenin and cyclin D1.

The same pattern, but even more marked, was seen in tumours treated with 17OH-pregnenolone.

β-AED did not reduce the expression of β-catenin or cyclin D1.

All steroids alike increased COX-2.

Exposure to steroid was 80 hours in all arms (contrary to the previous experiment where tumours were exposed for 96 hrs (α-AED) or 456 hours (β-AED and α+β-AED)).

Effects of α-AED, β-AED and 17αOH-Pregnenolone on Tumour Growth

An evaluation of the effects of sc. Injections in the proximity of the tumour as described in the section “Materials and Methods” above shows an approximate doubling of tumour volumes in β-AED-treated rats (200%) and untreated controls, an approximate 50% increase in rats treated with α-AED (150%) and an approximately halving (−50%) in animals treated with 17αOH-pregnenolone.

Effects on VEGF of α-AED, β-AED, 17αOH-Pregnenolone and 5-androstene-3β,7β,17α-Triol

Morphologic investigation of tumours treated with α-AED showed devitalized tumour tissue in circumscribed areas, giving the tumours a patchy appearance on microscopy. Onset of anti-tumoural effect was immediate. Considering the delayed onset of effect in β-AED treated tumours, and the very slow onset reported using troglitazone in vitro on human prostate cancer cell lines, another mechanism than increase of apoptosis through activation of PPARγ must be suspected.

Immunostaining as described in the section “Materials and Methods” above was performed in control tumours and 17α-AED treated tumours. A strong expression of Vascular Endothellal Growth Factor, VEGF, was demonstrated in controls ( 4/4) and a very weak in 17α-AED treated samples (¾).

Immunostaining was repeated for tumours from “animals II”. A strong staining for VEGF was demonstrated for controls ( 4/4) and 17β-AED (¾). An almost complete disappearance of staining for VEGF was demonstrated for ( 4/4) and 17αOH-pregnenolone ( 3/3). In tumours treated with 5-androstene-3β,7β,17α-triol (ββα-triol), there was an almost complete-disappearance of detectable VEGF in tumours in 2 cases, a modest downregulation in 1 after unchanged appearance in 1.

Results can be seen in FIG. 3 showing 5 μ sections of whole tumours where A. are tumour sections from untreated controls. Every second section is a negative control.

B. Are tumours treated with 17α-AED. Every second sample is a negative control.

C. Are tumours treated with 17β-AED. Every second sample is a negative control.

D. Are 5-androstene-3β,7β,17α-triol with three treated samples to the left followed by a negative control. To the right three samples treated with 17OH-pregnenolone, followed by a negative control.

Note superior growth inhibition in tumours treated with 17OH-pregnenolone. Also note lack of angiostatic effect in tumours treated with 17β-AED.

Comparison of Proportion of Cells in G1,S-Phase in Untreated Controls and After Treatment with 17α-AED

S-phase in untreated Dunning AT-1 samples shows a mean S-phase of 25.5% (24,24,24,30) compared to a mean S-phase of 45.3% in 17α-AED-treated samples (45,38,53,).

Proportion of cells in G1 was 59% in control tumours (55,52,65,67) and 35% (30,41,43) in treated tumours. Comparison of cells in G2 phase was difficult to estimate due to wide distribution of values.

FIG. 4 shows cell-cycle analysis for four different tumours treated with 17α-AED compared to four different tumours of untreated controls. Cell-cycle analysis is complicated by a considerable part of the cells being necrotic, causing a disturbance to interpretation of cells in G2.

PPARγ-Ligand Activation Assay

α- and β-androstenediols were investigated for PPARγ-ligand activity in a ligand-induced coactivator assay. No sign of transcription pointing to a ligand activity in the investigated androstenediols per se was seen.

Liver-cytosol incubated for two hours with α- or β-AED was investigated in ligand-induced coactivator assay. Weak bands with position corresponding to the positive control were present in liver cytosol. An identical result was seen when 17β-AED was added to the liver cytosol.

In the sample containing 17α-AED and liver cytosol the band was extinguished. When the experiment was repeated with 17α-AED, 3β,7β,17α-androstenetriol and 17α-OH-pregnenolone plus/minus PAPS and plus liver cytosol, which was also heat-inactivated and tested together with PAPS and 17α-AED, the result showed ligand activity only in the sample containing 17α-AED+PAPS and active liver cytosol (FIG. 5 ). The experiment is repeated with 17α-AED, 17β-AED±PAPS and liver cytosol ±heat inactivation by heating liver cytosol to 45° C. for 15 minutes to inactivate DHEA-sulfotransferase,

As positive controls SRC was used. No activity is demonstrated except in sample containing 17α-AED+PAPS and active liver cytosol.

FIG. 5 shows positive control (SRC-1) to the left, followed by negative control and then native steroids 17α-AED, 17OH-pregnenolone and 3β,7β,17α-androstenetriol+liver cytosol followed by steroids+liver cytosol+PAPS. A significant difference in signal is seen for the combination of 17α-AED+liver cytosol+PAPS. All other combinations, except positive control give insignificant responses.

Both α- and β-AED are naturally present in the human body. In adult humans in a proportion of 1:2, in the human fetus of 10:1. In the rat experiments, a ratio of 1:8 was used.

As mentioned above, in U.S. Pat. No. 5,912,240, Loria describes growth inhibition and apoptosis in monocytic tumour cell lines and growth inhibition in mammary cancer cell lines “independent of estrogen- or androgen receptors”. However, there is no suggestion as to the mechanism, and therefore, the practical applicability of said teachings of Loria is limited.

Further, in the above mentioned U.S. Pat. No. 5,912,240, it is suggested that antitumoural effects depend on an increase in programmed cell-death, apoptosis, in any type of malignant cell. However, the present inventors have been unable to confirm that effects of α-AED in rat prostatic tumour AT-1 in vivo is dependent on apoptosis. Likewise, no increased apoptosis in human androgen-refractory prostate cancer cell lines DU-145 or PC-3 exposed to 50-200 nM concentrations of 17α-AED could be shown by the present inventors. Quite contrary, an apoptosis inhibiting effect in these cell lines could be demonstrated. Since no connection could be made between a PPARγ-activity and the pure 17α-AED, the present inventors have rather contemplated a PPARγ-activity in dependence on 17α-AED-3βsulfate. The experiment was repeated with this compound, still without effect.

Discussing dependence of estrogen- or androgen-receptors, in the present application, tumour cells without a functioning androgen receptor in both PC-3 and DU-145 are discussed. Estrogen-receptor β (ERP) on the other hand is expressed in both cell lines. Estrogen-receptor α (ERα) only in PC-3. Treatment of both cell lines with ICI 172,780, which blocks both receptors gives a considerable growth inhibition and cell-death by itself in PC-3 cells.

This effect is greatly enhanced when 17α-AED-3βsulfate is added.

No obvious effect was seen in DU-145. The reason for this could be a non-functioning ERβ-signaling way, delayed or inhibited tumour cell death due to the existent P53-mutation in this cell type or the fact that DU-145 only has a very weak expression of PPARγ. Instead it expresses PPARδ, which is not regulated by the PPARγ-ligand 17α-AED-3βsulfate.

The fact that others present similar results on growth-inhibition for the same cell lines treated with thiazolidinediones and that the present inventors have been incapable to demonstrate a modulating effect of the present ligand on PPARγ-expression in PC-3, which is sensitive to treatment when estrogen-receptors are blocked, as well as a complete lack of modulating 5 effect of our ligand on the expression of PPARγ and δ in DU-145 in spite of complete ER-blockade speaks in favor of the latter argument.

Two breast cancer cell lines, the ER-positive MCF-7, with p53 wildtype and AR-positive and the ER-negative, p53-mutated SKBR-3, the latter with a strong expression of c-erbB2 were treated with 17α-AED-3βsulfate ±HER-2-antibodies.

Treatment of SKBR-3 with the same concentration used in the prostate cancer cell lines resuited in a modest increase in cell death. No effect was, however, seen in the ER+MCF-7, in spite of using 4 times higher concentrations than was done in U.S. Pat. No. 5,912,240. No additive effect from blocking of the c-erbB2-receptor with antibody was seen in any of the breast cancers. P53-mutation present in SKBR-3 did not prevent growth-inhibitory action from 17α-AED-3βsulfate. MCF-7, which is ER-positive but contains wild-type p53 continued to grow in spite of treatment with 17α-AED-3βsulfate. Taken together with the results of experiments with PC-3 and DU-145, this strongly suggests that 17α-AED-3βsulfate interacts with the estrogen-receptor, and that it is necessary to block this interaction with the estrogen-receptor when considering use in tissue expressing estrogen-receptor.

An anti-tumoural effect was demonstrated in Dunning rat prostate tumours in vivo. This effect is quick in onset, independent of effector cells of the immune system such as T-cells or macrophages and independent of apoptosis. Antitumoural effects are accompanied by devitalization in a patchy fashion and significant changes in VEGF expression in the tumour tissue. This strongly suggests an angiostatic mechanism. This was also demonstrated in microscopic sections of treated tumour. A further investigation also showed a downregulation of cyclin D1 and β-catenin. That this effect is neither exclusive to 17α-AED nor dependent on the PPARγ-activity that is tied to the metabolite, 17α-androstenediol-3β-sulfate, is supported by the finding that an even stronger antitumoural effect is seen when the same rat tumours are treated with 17αH-pregnenolone. This substance does not possess any PPARγ-activity, nor does its 3β-sulfate in order to extend this investigation further a new steroid, 5-androstene-3β,7β,17α-triol was constructed. There was no PPARγ-activity demonstrable in this steroid nor its 3β-sulfate. Down-regulatory effects on VEGF, γ-catenin and cyclin D1 were however demonstrated. An up-regulatory effect on plasminogen activator inhibitor 1 (PAI-1), which in turn inhibits plasminogen activator is a proposed mechanism for the effects of anglostatic steroids (Mechanism of action of angiostatic steroids: suppression of plasminogen activator activity via stimulation of plasminogen activator inhibitor synthesis: Blei F et al: J Cell Physiol 1993 June; 155(3): 568-78.).

The views of PPARγ-activity on angiogenesis differ. According to some evidence PAI-1 is down-regulated and PA thus up-regulated, speaking in favor of an angiogenic rather than angiostatic effect (Thiazolidinediones down-regulate PAI-1 expression in HUVEC: A possible role for PPARγ in endothelial function: Kato K et al: Biochem Biophys. Res. Commun.1999 May 10; 258(2): 431-5).

β-catenin is part of Wnt-signaling pathway and has been shown to influence cell cycle regulation and entry (Wnt-5a signaling in human mammary cells: Implications for the development of Breast Cancer: Marzieh Jönsson. Doctoral dissertation, Lund, September 2000).

The parallelism between antitumoural effects and lowering of β-catenin are suggestive of an influence on growth regulatory genes along this pathway such as cyclin D1, c-myc and c-met.

The importance of COX-2 in for instance colorectal cancers, but also in prostate cancer has been pointed out. The present results are contradictory concerning a possible co-regulation of COX-2 on the one side and cyclin D1, β-catenin and VEGF on the other. In tumours treated with the steroids mentioned, with simultaneous downregulatory effects on cyciln D1, β-catenin and VEGF, COX-2 is at the same time up-regulated, while at the same time a clear growth-inhibition of the tumour is observed. A close connection between PGE₁ and PGE₂ and an increased anglogenesis exists. (Prostaglandins up-regulate VEGF production through distinct pathways in differentiated U937 cells. Biochem. Biophys. Res. Commun. 2000 July5, 273(2); ₄₈₅-₉₁).

Thus, the divergent regulation of VEGF and COX-2 demonstrated by the present invention is highly surprising and unexpected.

In tumours treated with 17β-AED, a growth-stimulatory effect of this compound in tumour was observed for more than a week, before this was interrupted by an immunological antitumoural response. This effect is independent of estrogen- and androgen-receptor activation, since the used AT-1-tumour completely lacks such receptors. Since the first investigations of β-catenin and COX-2 expression in tumours treated with 17β-AED were not fully reliable, due to the fact that they were based on tumour investigation after 19 days of treatment, whereas the tumours treated with 17α-AED where treated only for 96 hours, the experiment was repeated with 17α-AED, 17β-AED,

17OH-pregnenolone and 5-androstene-3β,7β,17α-trlol. In this experiment all tumours were exposed to steroids for 80 hours before the rats were sacrificed. Western blotting showed no downregulatlon of COX-2 in spite of high expressions of β-catenin, VEGF and cyclin D1in tumours treated with 17β-AED.

Thus in spite of the inverse regulation of β-catenin, VEGF and cyclin DI on one side and COX-2 on the other, from the three angiostatic steroids, the opposite regulation is not found for COX-2 in tumours treated with 17β-AED. All four steroids up-regulate the expression of COX-2 when compared to untreated control tumours.

The usefulness of steroids S4 and S8 in prostate cancer of human or other origin with a functioning AR-signaling, which is usually the case even in cancers showing androgen-independent progression, is probably very limited if one considers use of 17α-AED or 17α-AAD in themselves since they are potential AR-ligands and easily converted into other potent AR-ligands.

For instance 17α-AED, which is converted into epitestosterone, dehydro-epiandrosterone, androstenedione or estradiol.
A) Epitestosterone will stimulate transcriptional activity in the AR, leading to disease progression.
B) β-catenin when up-regulated will enhance the effect of potential ligands on AR. Androstenedlone, androstenedlols, dehydro-epiandrosterone but also estradiol (all possible metabolites of 17α-AED) and the common androgen-receptor blockers as for instance bicalutamide have been demonstrated to increase transcriptional activity in AR, resulting in disease progression (β-catenin affects androgen-receptor activity and ligand specificity: Truica CI et al; Cancer Research. 60(17): 4709-13,2000 Sep.1.).
C) 17α-AED-3β-sulfate will be formed with increasing efficiency if the organism is deprived of other potential stimulators of androgen-receptor transcription. The mentioned AR-ligands will compete with the sulfate, which is a PPARγ-ligand for co-factors necessary for receptor-activation, among them SRC-1 and ARA-70. Overexpression of cyclin D1 is estimated to 4.2% of prostate cancers according to one source, but is probably more common than so, as androgen-independent progression through EGF-receptor stimulation, which is a common mechanism in disease progression, causes up-regulation of cyclin D1, (Overexpression of cyclin D1 is rare in human prostatic carcinoma: Gumbiner et al; Prostate. 38(1):40-5, Jan. 1.) (Epidermal growth factor induces cyclin D1 in a human prostate cancer cell line: Perry et al; Prostate.35(2):117-24,1998 May.).

In the above mentioned experiments by Waxman et al. (Role of metabolism in the activation of dehydro-epiandrosterone as a peroxisome proliferator. J. of Endocrinology vol150, suppl, Sept 1996), indirect evidence of PPARα activity was demonstrated in 17β-AED in vivo, but not in vitro, and it was also demonstrated that the 3β-sulfate of 17β-AED or DHEA was more efficient than corresponding native steroid in activating genes connected to PPARα. As the androstanediols are closely related chemically it seems likely that they have a parallelism in metabolic pathways and the hypothesis that PPARγ-ligand activity is dependent on the 3β-sulfate of 17α-AED was suggested by the present inventors. This hypothesis is supported by the findings that ligand activity is dependent on a sulfate donor, PAPS and that liver cytosol, which is the main source of steroid sulphotransferase, needs to be present. The DHEA sulphotransferase catalyzes a transfer of sulfate specifically to the 3β-position in 3β-OH-steroids.

No increased cell-death, apoptotic or necrotic, was seen on exposing cell lines DU-145 or PC-3 to 100 or 200 nM concentrations of 17α-androstenediol in vitro in spite of investigating cells by means of expression of caspase 3 or 7. Staining with Evans Blue in order to differentiate between vital and dead cells also did not reveal any increase in proportion of dead cells in treated samples. Comparing cell numbers in culture flasks treated with 17α-AED with untreated controls at regular time intervals showed no decrease in cell number in cultures treated with 17α-AED, speaking against a growth inhibitory effect of 17α-AED in itself in these cell lines.

The present failure to show any increased number of devitalized cells in the trypan blue exclusion trial supports these findings.

Experiments in vitro with TZD in human prostatic cancer cell-lines PC-3, LNCaP and DU-145 demonstrated a pronounced antitumoural effect in PC-3, an intermediate effect in LNCaP and no effect in DU-145 (Ligand for PPARγ (Troglitazone) has potent antitumour effect against human prostate cancer both in vitro and in vivo: Kubota et al: Cancer Research 58,3344-3352, Ayg 1,1998). This effect took however considerable time. That no antitumoural effect was noted in DU-145 in the present experiments is in accordance with the experiments with TZD, cited above. An explanation for this lack of effect is the present finding of a very weak expression of PPARγ in DU-145 cells, whereas PPARδ is strongly expressed. No change in PPARγ or PPARδ expression was observed in cultures treated with 17α-AED.

In other experiments with PPARγ-ligands and the same human prostate cancer cell lines, increased cell death was demonstrated using electron microscopy. This cell death was of a non-apoptotic nature (Non-apoptotic cell death associated with S-phase arrest of prostate cancer cells via the PPARγ-ligand 15-deoxy-Δ 12,14-prostaglandin J2: Butler et al: Cell Growth & Differentiation, Vol.11, 49-61, January 2000). In the receptor assay, no ligand-activation was seen when using 17α or 17β-AED as single substances. Not unexpectedly, a weak PPARγ-ligand activity was present in the liver cytosol and this activity was unaltered when 17β-AED was added but extinguished when 17α-AED replaced the 17β-epimer. Taken together, this shows that both epimers lack PPARγ agonistic properties and suggests the possibility of a PPARγ antagonistic function for 17α-AED,

Upregulation of PPARγ in Dunning AT-1, rat prostatic cancer in vivo, treated with 17α-AED and the differentiation seen on prolonged exposure as well as the down-regulation of COX-2, an effect expected from a PPARγ-ligand suggest that the PPARγ activating capacity rather than depending on 17α-AED itself is tied to a metabolite, the sulfated form.

The unexpected results seen in Western blotting, where 17α-AED results in an up-regulation of PPARγ-activity and at the same time of an up-regulation of COX-2 at the same time as β-catenin is down-regulated by 17α-AED as well as 17β-AED, might be explained in the following way: As a result of sulphotransferase activity in viva, 17α-AED is sulfated, explaining the up-regulation of PPAR. 17β-AED is sulfated in the same manner. As the sulfated form of 17β-AED is a PPARα activating compound, an increase in β-catenin as well as c-myc, c-met and cyclin D1 would be expected (Effect on the expression of c-met, c-myc and PPAR-alpha in liver and liver tumours from rats chronically exposed to the hepatocarcinogenic peroxisome proliferator WY-14,643: (Miller RT et al., Carcinogenesis 1996 June;17(6):1337-41). In accordance with this expectation one would expect an increased growth rate, which is indeed what is found in Dunning AT-1 tumours exposed to 17β-AED for 96 hours. This is one of the reasons that the animal experiment was repeated, and that a uniform exposure time to steroids of 80 hours for all tumours was chosen.

In accordance with the present theory, Western blotting of these tumours showed that exposure to 17β-AED does not result in a down regulation of β-catenin, nor to cyclin D1. The down-regulation seen on exposure to 17α-AED, 17hydroxy-pregnenolone as well as 5-androstene-3β,7β,17α-triol, the latter due to substitution in 7- position, not readily convertible to 17α-AED and hence not into its 3β-sulfate, shows that the effect is independent of PPARγ, since the latter two substances are not converted to a PPARγ-ligand when sulfated. It is possible that the diminished tumour seen after prolonged exposure to 17β-AED reflects a devitalized tumour containing mainly stromal cells (which seemed to be the case in microscopic viewing) and accordingly less β-catenin.

The down-regulation of COX-2 seen when 17β-AED and 17α-AED are combined can either be a result of combined PPARα and γ-activation or a result of PPARγ-activation.

In spite of a doubling also of COX-2, with known angiogenic properties a downregulatlon of VEGF and an antitumoural effect was observed in tumours exposed to 17α-AED. That expression of PPARγ in itself does not lead to an antitumoural effect was demonstrated through the lack of antitumoural effect in PC-3 cell line of the 3β-sulfate. The lack of effect suggests a binding of 17α-AED to estrogen-receptors, as a growth inhibition and cell death occurs only when estrogen-receptors are blocked. In experiments with Saccharomyces species transfected with an androgen receptor (AR) it was shown that co-factor ARA-70 plays an essential role when 17β-AED is bound to this receptor and activates it. ARA-70 plays the role of co-factor also in PPARγ, were it amplifies the receptor-ligand response, but also is able to activate the receptor by itself. A simultaneous presence of AR is able to quench the activated PPARγ-ligand complex, suggesting a competition for co-factor.

The influence of ARA-70 in ER is neglectible. (Identification of ARA70 as a ligand-enhanced coactivator for the PPARγ: Heinlein Cynthia et al: J. Of Biol. Chemistry, vol 274, No23, June4, 16147-16152,1999). This points to some foreseeable effects in an androgen-dominated system like the prostate gland.

1. Presence of testosterone, dihydrotestosterone and other ligands to AR, such as 17β-AED and possibly 17α-AED, or at least its metabolite, epitestosterone are likely to counteract the effects of PPARγ-ligands. Flutamide and bicalutamide as means to block AR-activation are doubtful or at least incomplete as androgenic properties are activated in these substances in the presence of ARA-70 as well as in cases of up-regulated β-catenin.

Androgen receptors are expressed in many organs. What is known of the down-regulation of the receptor is that It is partly influenced by ERα, which has a down-regulatory effect. It is of course influenced by diminished access to ligands, such as testosterone or dihydrotestosterone. It is also downregulated by resveratrol and by activation of the Arylhydrocarbonreceptor.

Other activating influences on the androgen-receptor are signaling through EGF or HER-2-receptors as well as interleukin-6 (IL-6). Influences that It might be necessary to block. Signaling through IL-6 is counteracted through PPARγ though. The other factors could be blocked through antibodies or through use of melatonin, which stops the production of EGF-R ligands such as hydroxylinoleic acids.

2. The immunoenhancing effects behind the antitumoural action of 17β-AED are counteracted by PPARγ-ligands as activation of this receptor gives many of the effects that characterizes IL-4, a cytokine resulting in a T-helper 2 type response, the very opposite to what is useful in tumour immunotherapy. The concept to use sequential treatment with first 17α-AED followed by 17β-AED is thus not useful at all to achieve an antitumoural effect. As was demonstrated in the original experiment this combination also lacked antitumoural effects, but even stimulated tumour-growth.

3. The presence of ERα and ERβ calls for an effective receptor blockade. Tamoxifen or raloxifen can be used for this purpose. From what is known today about the controlling function of ERA there is no need to control the activity of this receptor as this is the receptor that suppresses ERα-activity through its ligand-3β,17β-androstanediol. In a tumour system, this does not necessarily hold true, as cell-signaling might be defect.

An up-regulation of VEGF through PDGF-BB in turn conferred through effects on c-fos from endothelial cells, which are known to express PPARγ have been described (PPARγ agonists increase VEGF expression in human vascular smooth muscle cells: Yamakawa K et al, Biochem. Biophys. Res. Commun. 2000 May19, 271(3):571-4). Decrease in MMP-9 was also observed after treatment with PPARγ-ligands. Publications mainly report an upregulation of VEGF through PPARγ. A strong upregulation of VEGF in p53-mutated tumours is also reported.

In summary, the present invention provides evidence to different mechanisms behind the growth-inhibition observed in some neoplastic cell lines and the antitumoural effects seen in vivo in Dunning AT-1 rat prostatic cancer.

In the first place a conversion of 17α-AED to sulfated form through the action of a sulphotransferase is responsible for inhibition of cell growth, apoptosis or cell cycle arrest. This is provided through an activation of PPARγ.

Effects of this type are mainly dependent on sulfotransferase activity and preferably DHEA-sulfotransferase, which is present in the liver, In adrenals, in testes and in small intestine. Its presence in placenta is not yet documented, but the related pregnenolone-sulfotransferase is present. It is likely that a sulfotransferation with much lower substrate specificity occurs also elsewhere, especially with pregnenolone-sulfotransferase and estrogen-sulfotransferase, the latter being present in many tissues. In order to maintain the PPARγ-activating effect of 17α-AEDS, it is of importance to diminish sulfatase activity in the organ to be treated. This is otherwise a process that will deactivate 17α-AEDS. This can be avoided through the administration of a proper quantity of Coumateo, which is a non-estrogenic inhibitor of sulfatase.

The present inventors have demonstrated growth inhibition in 3T3L1 fibroblasts and in ER-negative breast cancer cell line SKBR-3 treated with 17α-AED-3β-sulfate. In human androgen-refractory prostate cancer cell lines PC-3 and DU-145, a considerable cell death in PC-3 cells was shown when estrogen-receptors were blocked. No such effect was however seen in DU-145. The latter is possibly dependent on very low expression of PPARγ in this cell line, which is instead dominated, by a strong expression of PPAR5.

Other tumours known to express PPARγ and hence expected to respond to 3β-sulfate of 17α-AED, are urothelial cancers, gastric cancers, malignancies derived from endothelial cells, smooth muscle cells, cancer of the colon, chorioncarcinomas, adenocarcinomas of the lung, gastric cancers and liposarcomas as well as several aspects of pathology of the eye tissues, such as cells of the macula and glaucoma which are all influenced by therapy with PPARγ-ligands. Monocytes and lymphocytes are downregulated in their production of proinflammatory cytokines (IL-1, TNF-α and IL-6) as well as cytokines of Thelper1-profile in T-cells (IFN-γ, TNF-β and IL-2). Instead IL-4-effects are promoted consistent with a stimulation of a T-helper 2-profile. This also includes inflammatory bowel diseases such as Crohn's disease, diseases of the placental tissue, autoimmune, inflammatory diseases such as rheumatoid arthritis, neurodegenerative diseases such as multiple sclerosis and Guillain-Barres syndrome and many others.

The second mechanism of action is cell cycle regulatory and antiangiogenic and not mediated through PPAPγ, as demonstrated by an even stronger antitumoural effect from 17OH-pregnenolone than 17α-AED,

The present observation of a downregulation of β-catenin after treatment with 17α- AED absent in 17β-AED in rat prostate cancer suggests that the two androstenediol epimers might be a regulatory pair in this respect.

That this effect in 17α-AED also exists in 17αOH-pregnenolone and 5-androstene-3β,7β,17α-triol suggests that antitumoural effects of these substances are independent of PPARγ, since sulfate of the latter substances lack PPARγ-ligand activity. All three substances very markedly downregulate β-catenin, a factor of importance for tumour growth. An increased expression of β-catenin might explain the growth stimulation initially seen in rat prostate cancer in vivo, exposed to 17β-AED. Effects through estrogen- or androgenreceptors are insufficient as explanation, since Dunning AT-1 rat prostatic cancer lacks measurable quantities of these receptors.

Effects in other tumours through effects on cell cycle entry and angiogenesis are also encompassed by the present invention,

Thus, when an inhibition of growth and an angiostatic effect is desired, 17βH-pregnenolone or some other steroid, which is not, directly stimulating PPARγ through conversion into sulfate is chosen. Through use of uncastrated animals in an androgenic environment the risk for PPARγ-activity is further diminished, through competition of cofactors (quenching). Androgens also block sulphotransferase activity. If on the other hand PPARγ-activatlon is desired, then androgen should be minimized, sulfatase activity should be kept down through an inhibitor such as Coumate® and tumours with increased expression of β-catenin or cyclin D1 should be avoided. The latter a possible sign have increased EGF-R signaling.

The present data shows that the present compounds are potential therapeutically active compounds when it comes to treatment of tumours, benign as well as malignant ones.

The present invention further encompasses prodrugs of the compounds of the invention, whereby such prodrugs encompass esters as well as other prodrugs encompassing protecting groups on the hydroxy-groups, which protecting groups are cleaved off during metabolism.

The present compounds are administered in therapeutically effective amounts, and preferably in such amounts as to reach a blood serum concentration of 50 to 500 nM.

Pharmaceutical compositions of the invention are prepared in the form of granules, tablets, or Injectable solution, containing the active compound together with one or more therapeutically inert excipients. The compositions may comprise from 0.5 to 99.5% by weight of the active drug.

In order to obtain prolonged release of the compounds they may be administered in combination with cationic dextranes, as well as they can be administered in the form of amino substituted compounds, wherein one or more of carbon numbers 7, 11, and 16 may be substituted.

1-33. (canceled)

34. A method for treating disturbances in Wnt-signaling, cell cycle arrest in G1-phase and/or providing an angiostatic effect by administering a therapeutically effective amount of a steroid derivative selected from a group of compounds defined by formulas 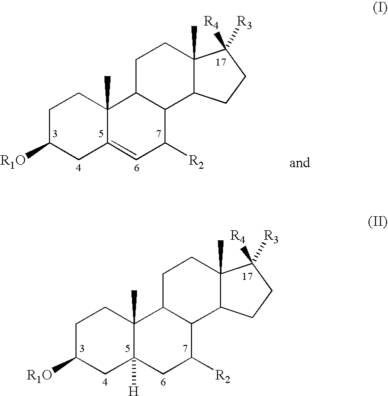
having a substituent R₁O in 3beta-position, R₃ in 17alpha-position, and R₄ in 17beta-position, wherein the only difference between said formulas (I) and (II) is the bond between carbon number 5 and carbon number 6,
wherein R₁O is in beta-position and R₁ is H, or a protecting group, in the form of CH₃, CH₂OMe, or CH₂O-alkyl;
R₂ in beta-position O═—OH;
R₃ in alpha-position and is a hydroxyl group, an acyl group or an alkoxy group R″0, wherein R″ has the same meaning as R₁, and
R₄ in beta-position is H, an alkyl, an acyl, or an alkoxy group, said steroid derivative capable of interrupting Wnt-signaling especially in cases where said Wnt-signaling is disturbed as by elevated levels of beta-catenin or cyclin D1, interrupting cell cycle arrest in G-1 phase and/or providing an angiostatic effect.

35. (canceled)

36. (canceled)

37. The method according to any one of claims 33-35, wherein said interruption of Wnt-signaling is provided by down-regulating beta-catenin or cyclin D1 by the administration of said steroid derivative.

38. The method using a steroid derivative according to any one of claims 33-35, wherein the steroid derivative is administered in a therapeutically effective amount to prevent and/or counteract overexpression of factors present in the Wnt-signaling pathway in tumour systems with a phenotypic or genotypic deviance in this respect, such as mammary carcinomas, lung cancers, head and neck cancers, melanomas, and esophageal cancers and others.

39. The method according to claim 38, wherein said steroid is selected from the group consisting of 5-androstane-3,17-diol, 17-hydroxy-pregnenolone and 17-hydroxy-pregnanolone.

40. A method for treating non-tumour conditions in the form of neovascularisation or excessive growth of fibroblasts, in the form of hypertrophic scars, keloids, corneal neovascularisation, diabetic retinopathy or exsudative forms of macular degeneration by administering a therapeutically effective amount of a steroid derivative selected from the group consisting of 17-hydroxy-pregnenolone, 5-androstene-3beta, 17alpha-diol 17-hydroxy-pregnanolone, 5-androstane-3beta, 17alpha-diol or esters or ethers of these steroids.

41-42. (canceled)

43. The method according to claim 34, wherein said steroid derivative is selected from the group consisting of 5-androstene-3,7,17-triol, 5-androstene-3, 17- diol-7-one, androstane-3,7,17 -trioi and androstane-3,17-diol-7-one, capable of interrupting disturbances in Wnt-signalling,cell cycle arrest in G1-phase and/or providing an angiostatic effect.

44. The method according to claims 34 and 35 wherein the substituent R₄ is an acyl group where H, alkoxy, alkyl, alkenyl or alkinyl is attached to the keto group carbon.
